(19) United States
(12) Patent Application Publication
(10) Pub. No.: US 2016/0022684 A1
(43) Pub. Date: Jan. 28, 2016

(54) BET INHIBITOR AND BRUTON'S TYROSINE KINASE INHIBITOR COMBINATIONS

(71) Applicant: Pharmacyclics LLC, Sunnyvale, CA (US)

(72) Inventors: Hsu-Ping KUO, Sunnyvale, CA (US); Hsin-Kang HSIEH, San Jose, CA (US); Betty CHANG, Cupertino, CA (US)

(21) Appl. No.: 14/807,680

(22) Filed: Jul. 23, 2015

Related U.S. Application Data
(60) Provisional application No. 62/029,346, filed on Jul. 25, 2014.

(51) Int. Cl. A61K 31/519; G01N 33/574; A61K 45/06; C12Q 1/68; A61K 31/4745; A61K 31/551

(52) U.S. Cl. CPC A61K 31/519; A61K 31/4745; A61K 31/551; A61K 45/06; C12Q 1/6886; G01N 33/57426; C12Q 2600/158

(57) ABSTRACT

Disclosed herein are methods, compositions, and kits for treating a B-cell malignancy comprising administering a combination of a BTK inhibitor (e.g., ibrutinib) and a BET inhibitor. Also disclosed herein are methods, compositions, and kits for treating a BTK-resistant B cell malignancy, or a MYC-driven B cell malignancy comprising administering a combination of a BTK inhibitor (e.g., ibrutinib) and a BET inhibitor. Further disclosed herein are methods of evaluating a patient having a B-cell malignancy for treatment with a combination of a BTK inhibitor (e.g., ibrutinib) and a BET inhibitor based on the MYC expression level of the patient.

### CROSS-REFERENCE TO RELATED APPLICATIONS

This application claims the benefit of priority from U.S. Provisional Application No. 62/029,346, filed Jul. 25, 2014, which application is incorporated herein by reference.

### BACKGROUND OF THE INVENTION

Bruton's tyrosine kinase (BTK), a member of the Tec family of non-receptor tyrosine kinases, is a key signaling enzyme expressed in all hematopoietic cells types except T lymphocytes and natural killer cells. BTK plays an essential role in the B-cell signaling pathway linking cell surface B-cell receptor (BCR) stimulation to downstream intracellular responses.

### SUMMARY OF THE INVENTION

Disclosed herein, in certain embodiments, are methods of treating a B-cell malignancy in a subject in need thereof, comprising administering to the subject a therapeutically effective amount of a combination comprising a BTK inhibitor and a BET inhibitor. In some embodiments, the combination provides a synergistic therapeutic effect compared to administration of the BTK inhibitor or the BET inhibitor alone. In some embodiments, the combination sensitizes a B-cell malignancy to the BTK inhibitor. In some embodiments, the BET inhibitor comprises CPI-0610, DUAL946, GSK525762, I-BET151, JQ1, OTX015, PFI-1, RVX-208, RVX2135, TEN-010, or a combination thereof. In some embodiments, the BET inhibitor is I-BET151, JQ1, OTX015, or a combination thereof. In some embodiments, the BET inhibitor is I-BET151. In some embodiments, the BET inhibitor is JQ1. In some embodiments, the BET inhibitor is OTX015. In some embodiments, the BTK inhibitor is a compound of Formula (A) or Formula (A1). In some embodiments, the BTK inhibitor is ibrutinib. In some embodiments, the BTK inhibitor is ibrutinib and the BET inhibitor is selected from among CPI-0610, DUAL946, GSK525762, I-BET151, JQ1, OTX015, PFI-1, RVX-208, RVX2135, TEN-010, or a combination thereof. In some embodiments, the BTK inhibitor is ibrutinib and the BET inhibitor is I-BET151, JQ1, OTX015, or a combination thereof. In some embodiments, the BTK inhibitor is ibrutinib and the BET inhibitor is I-BET151. In some embodiments, the BTK inhibitor is ibrutinib and the BET inhibitor is JQ1. In some embodiments, the BTK inhibitor is ibrutinib and the BET inhibitor is OTX015. In some embodiments, the B-cell malignancy is acute lymphoblastic leukemia (ALL), acute myelogenous leukemia (AML), chronic myelogenous leukemia (CML), acute monocytic leukemia (AMoL), chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is diffuse large B-cell lymphoma (DLBCL). In some embodiments, the DLBCL is activated B-cell diffuse large B-cell lymphoma (ABC-DLBCL). In some embodiments, the B-cell malignancy is a relapsed or refractory B-cell malignancy. In some embodiments, the B-cell malignancy is a BTK inhibitor-resistant B cell malignancy. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, ibrutinib and the BET inhibitor are administered simultaneously, sequentially or intermittently. In some embodiments, the method further comprises administering a third therapeutic agent. In some embodiments, the third therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, are methods of treating a BTK inhibitor-resistant B cell malignancy comprising administering to a subject in need thereof a therapeutically effective amount of a combination comprising a BTK inhibitor and a BET inhibitor. In some embodiments, the combination provides a synergistic therapeutic effect compared to administration of the BTK inhibitor or the BET inhibitor alone. In some embodiments, the combination sensitizes a B-cell malignancy to the BTK inhibitor. In some embodiments, the BET inhibitor comprises CPI-0610, DUAL946, GSK525762, I-BET151, JQ1, OTX015, PFI-1, RVX-208, RVX2135, TEN-010, or a combination thereof. In some embodiments, the BET inhibitor is I-BET151, JQ1, OTX015, or a combination thereof. In some embodiments, the BET inhibitor is I-BET151. In some embodiments, the BET inhibitor is JQ1. In some embodiments, the BET inhibitor is OTX015. In some embodiments, the BTK inhibitor is a compound of Formula (A) or Formula (A1). In some embodiments, the BTK inhibitor is ibrutinib. In some embodiments, the BTK inhibitor is ibrutinib and the BET inhibitor is selected from among CPI-0610, DUAL946, GSK525762, I-BET151, JQ1, OTX015, PFI-1, RVX-208, RVX2135, TEN-010, or a combination thereof. In some embodiments, the BTK inhibitor is ibrutinib and the BET inhibitor is I-BET151, JQ1, OTX015, or a combination thereof. In some embodiments, the BTK inhibitor is ibrutinib and the BET inhibitor is I-BET151. In some embodiments, the BTK inhibitor is ibrutinib and the BET inhibitor is JQ1. In some embodiments, the BTK inhibitor is ibrutinib and the BET inhibitor is OTX015. In some embodiments, the B-cell malignancy is acute lymphoblastic leukemia (ALL), acute myelogenous leukemia (AML), chronic myelogenous leukemia (CML), acute monocytic leukemia (AMoL), chronic lymphocytic leukemia (CLL), high-risk CLL, small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is diffuse large B-cell lymphoma (DLBCL). In some embodiments, the DLBCL is activated B-cell diffuse large B-cell lymphoma (ABC-DLBCL). In some embodiments, the B-cell malignancy is a relapsed or refractory B-cell malignancy. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, ibrutinib and the BET inhibitor are administered simultaneously, sequentially or intermittently. In some embodiments, the method further comprises administering a third therapeutic agent. In some embodiments, the third therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, are methods of treating a diffuse large B-cell lymphoma (DLBCL) comprising administering to a subject in need thereof a therapeutically effective amount of a combination comprising a BTK inhibitor and a BET inhibitor. In some embodiments, the combination provides a synergistic therapeutic effect compared to administration of the BTK inhibitor or the BET inhibitor alone. In some embodiments, the combination sensitizes a B-cell malignancy to the BTK inhibitor. In some embodiments, the BET inhibitor comprises CPI-0610, DUAL946, GSK525762, I-BET151, JQ1, OTX015, PFI-1, RVX-208, RVX2135, TEN-010, or a combination thereof. In some embodiments, the BET inhibitor is I-BET151, JQ1, OTX015, or a combination thereof. In some embodiments, the BET inhibitor is I-BET151. In some embodiments, the BET inhibitor is JQ1. In some embodiments, the BET inhibitor is OTX015. In some embodiments, the BTK inhibitor is a compound of Formula (A) or Formula (A1). In some embodiments, the BTK inhibitor is ibrutinib. In some embodiments, the BTK inhibitor is ibrutinib and the BET inhibitor is selected from among CPI-0610, DUAL946, GSK525762, I-BET151, JQ1, OTX015, PFI-1, RVX-208, RVX2135, TEN-010, or a combination thereof. In some embodiments, the BTK inhibitor is ibrutinib and the BET inhibitor is I-BET151, JQ1, OTX015, or a combination thereof. In some embodiments, the BTK inhibitor is ibrutinib and the BET inhibitor is I-BET151. In some embodiments, the BTK inhibitor is ibrutinib and the BET inhibitor is JQ1. In some embodiments, the BTK inhibitor is ibrutinib and the BET inhibitor is OTX015. In some embodiments, the DLBCL is activated B-cell diffuse large B-cell lymphoma (ABC-DLBCL). In some embodiments, the DLBCL is a relapsed or refractory DLBCL. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, ibrutinib and the BET inhibitor are administered simultaneously, sequentially or intermittently. In some embodiments, the method further comprises administering a third therapeutic agent. In some embodiments, the third therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, are methods of treating a B-cell malignancy associated with an elevated expression of c-MYC, comprising: (a) determining the expression level of c-MYC in a sample from an individual; and (b) administering to the individual a therapeutically effective amount of a combination comprising a BTK inhibitor and a BET inhibitor if the individual has an elevated expression level of c-MYC. In some embodiments, the elevated level of c-MYC is 1-fold, 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher compared to the expression level of the reference. In some embodiments, the reference level is the expression level of c-MYC in an individual who does not have a B-cell malignancy. In some embodiments, the combination provides a synergistic therapeutic effect compared to administration of the BTK inhibitor or the BET inhibitor alone. In some embodiments, the combination sensitizes a B-cell malignancy to the BTK inhibitor. In some embodiments, the BET inhibitor comprises CPI-0610, DUAL946, GSK525762, I-BET151, JQ1, OTX015, PFI-1, RVX-208, RVX2135, TEN-010, or a combination thereof. In some embodiments, the BET inhibitor is I-BET151, JQ1, OTX015, or a combination thereof. In some embodiments, the BET inhibitor is I-BET151. In some embodiments, the BET inhibitor is JQ1. In some embodiments, the BET inhibitor is OTX015. In some embodiments, the BTK inhibitor is a compound of Formula (A) or Formula (A1). In some embodiments, the BTK inhibitor is ibrutinib. In some embodiments, the BTK inhibitor is ibrutinib and the BET inhibitor is selected from among CPI-0610, DUAL946, GSK525762, I-BET151, JQ1, OTX015, PFI-1, RVX-208, RVX2135, TEN-010, or a combination thereof. In some embodiments, the BTK inhibitor is ibrutinib and the BET inhibitor is I-BET151, JQ1, OTX015, or a combination thereof. In some embodiments, the BTK inhibitor is ibrutinib and the BET inhibitor is I-BET151. In some embodiments, the BTK inhibitor is ibrutinib and the BET inhibitor is JQ1. In some embodiments, the BTK inhibitor is ibrutinib and the BET inhibitor is OTX015. In some embodiments, the B-cell malignancy is acute lymphoblastic leukemia (ALL), acute myelogenous leukemia (AML), chronic myelogenous leukemia (CML), acute monocytic leukemia (AMoL), chronic lymphocytic leukemia (CLL), high risk CLL, small lymphocytic lymphoma (SLL), high-risk SLL, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is diffuse large B-cell lymphoma (DLBCL). In some embodiments, the DLBCL is activated B-cell diffuse large B-cell lymphoma (ABC-DLBCL). In some embodiments, the B-cell malignancy is a relapsed or refractory B-cell malignancy. In some embodiments, the sample is a blood sample or a serum sample. In some embodiments, determining the expression level of c-MYC in the sample comprises measuring the amount of nucleic acid encoding c-MYC in the sample. In some embodiments, the sample comprises one or more tumor cells. In some embodiments, the nucleic acid is mRNA. In some embodiments, the method further comprises detecting the nucleic acid using a microarray. In some embodiments, the method further comprises amplification of the nucleic acid. In some embodiments, the amplification is a polymerase chain reaction. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, ibrutinib and the BET inhibitor are administered simultaneously, sequentially or intermittently. In some embodiments, the method further comprises administering a third therapeutic agent. In some embodiments, the third therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, is a method of selecting an individual having a B-cell malignancy for therapy with a combination comprising a BTK inhibitor and a BET inhibitor, comprising: (a) measuring the expression level of c-MYC in a sample from the individual; (b) comparing the expression level of c-MYC with a reference level; and (c) characterizing the individual as a candidate for therapy with the combination comprising a BTK inhibitor and a BET inhibitor if the individual has an elevated level of c-MYC compared to the reference level. In some embodiments, the elevated level of MYC is 1-fold, 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher compared to the expression of the reference level. In some embodiments, the reference level is the expression level of c-MYC in an individual who does not have a B-cell malignancy. In some embodiments, the combination provides a synergistic therapeutic effect compared to administration of the BTK inhibitor or the BET inhibitor alone. In some embodiments, the combination sensitizes a B-cell malignancy to the BTK inhibitor. In some embodiments, the BET inhibitor comprises CPI-0610, DUAL946, GSK525762, I-BET151, JQ1, OTX015, PFI-1, RVX-208, RVX2135, TEN-010, or a combination thereof. In some embodiments, the BET inhibitor is I-BET151, JQ1, OTX015, or a combination thereof. In some embodiments, the BET inhibitor is I-BET151. In some embodiments, the BET inhibitor is JQ1. In some embodiments, the BET inhibitor is OTX015. In some embodiments, the BTK inhibitor is a compound of Formula (A) or Formula (A1). In some embodiments, the BTK inhibitor is ibrutinib. In some embodiments, the BTK inhibitor is ibrutinib and the BET inhibitor is selected from among CPI-0610, DUAL946, GSK525762, I-BET151, JQ1, OTX015, PFI-1, RVX-208, RVX2135, TEN-010, or a combination thereof. In some embodiments, the BTK inhibitor is ibrutinib and the BET inhibitor is I-BET151, JQ1, OTX015, or a combination thereof. In some embodiments, the BTK inhibitor is ibrutinib and the BET inhibitor is I-BET151. In some embodiments, the BTK inhibitor is ibrutinib and the BET inhibitor is JQ1. In some embodiments, the BTK inhibitor is ibrutinib and the BET inhibitor is OTX015. In some embodiments, the B-cell malignancy is acute lymphoblastic leukemia (ALL), acute myelogenous leukemia (AML), chronic myelogenous leukemia (CML), acute monocytic leukemia (AMoL), chronic lymphocytic leukemia (CLL), high risk CLL, small lymphocytic lymphoma (SLL), high risk SLL, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is diffuse large B-cell lymphoma (DLBCL). In some embodiments, the DLBCL is activated B-cell diffuse large B-cell lymphoma (ABC-DLBCL). In some embodiments, the B-cell malignancy is a relapsed or refractory B-cell malignancy. In some embodiments, the sample is a blood sample or a serum sample. In some embodiments, determining the expression level of c-MYC in the sample comprises measuring the amount of nucleic acid encoding c-MYC in the sample. In some embodiments, the sample comprises one or more tumor cells. In some embodiments, the nucleic acid is mRNA. In some embodiments, the method further comprises detection the nucleic acid using a microarray. In some embodiments, the method further comprises amplification of the nucleic acid. In some embodiments, the amplification is a polymerase chain reaction.

Disclosed herein, in certain embodiments, are pharmaceutical combinations comprising: (a) a BTK inhibitor; (b) a BET inhibitor; and (c) a pharmaceutically-acceptable excipient. In some embodiments, the combination provides a synergistic therapeutic effect compared to administration of the BTK inhibitor or the BET inhibitor alone. In some embodiments, the combination sensitizes a B-cell malignancy to the BTK inhibitor. In some embodiments, the BET inhibitor comprises CPI-0610, DUAL946, GSK525762, I-BET151, JQ1, OTX015, PFI-1, RVX-208, RVX2135, TEN-010, or a combination thereof. In some embodiments, the BET inhibitor is I-BET151, JQ1, OTX015, or a combination thereof. In some embodiments, the BET inhibitor is I-BET151. In some embodiments, the BET inhibitor is JQ1. In some embodiments, the BET inhibitor is OTX015. In some embodiments, the BTK inhibitor is a compound of Formula (A) or Formula (A1). In some embodiments, the BTK inhibitor is ibrutinib. In some embodiments, the BTK inhibitor is ibrutinib and the BET inhibitor is selected from among CPI-0610, DUAL946, GSK525762, I-BET151, JQ1, OTX015, PFI-1, RVX-208, RVX2135, TEN-010, or a combination thereof. In some embodiments, the BTK inhibitor is ibrutinib and the BET inhibitor is I-BET151, JQ1, OTX015, or a combination thereof. In some embodiments, the BTK inhibitor is ibrutinib and the BET inhibitor is I-BET151. In some embodiments, the BTK inhibitor is ibrutinib and the BET inhibitor is JQ1. In some embodiments, the BTK inhibitor is ibrutinib and the BET inhibitor is OTX015. In some embodiments, the combination is in a combined dosage form. In some embodiments, the combination is in separate dosage forms.

Disclosed herein, in certain embodiments, are uses of a therapeutically effective amount of a combination comprising a BTK inhibitor and a BET inhibitor for treating a B-cell malignancy in a subject in need thereof. In some embodiments, the combination provides a synergistic therapeutic effect compared to administration of the BTK inhibitor or the BET inhibitor alone. In some embodiments, the combination sensitizes a B-cell malignancy to the BTK inhibitor. In some embodiments, the BET inhibitor comprises CPI-0610, DUAL946, GSK525762, I-BET151, JQ1, OTX015, PFI-1, RVX-208, RVX2135, TEN-010, or a combination thereof. In some embodiments, the BET inhibitor is I-BET151, JQ1, OTX015, or a combination thereof. In some embodiments, the BET inhibitor is I-BET151. In some embodiments, the BET inhibitor is JQ1. In some embodiments, the BET inhibitor is OTX015. In some embodiments, the BTK inhibitor is a compound of Formula (A) or Formula (A1). In some embodiments, the BTK inhibitor is ibrutinib. In some embodiments, the BTK inhibitor is ibrutinib and the BET inhibitor is selected from among CPI-0610, DUAL946, GSK525762, I-BET151, JQ1, OTX015, PFI-1, RVX-208, RVX2135, TEN-010, or a combination thereof. In some embodiments, the BTK inhibitor is ibrutinib and the BET inhibitor is I-BET151, JQ1, OTX015, or a combination thereof. In some embodiments, the BTK inhibitor is ibrutinib and the BET inhibitor is I-BET151. In some embodiments, the BTK inhibitor is ibrutinib and the BET inhibitor is JQ1. In some embodiments, the BTK inhibitor is ibrutinib and the BET inhibitor is OTX015. In some embodiments, the B-cell malignancy is acute lymphoblastic leukemia (ALL), acute myelogenous leukemia (AML), chronic myelogenous leukemia (CML), acute monocytic leukemia (AMoL), chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is diffuse large B-cell lymphoma (DLBCL). In some embodiments, the DLBCL is activated B-cell diffuse large B-cell lymphoma (ABC-DLBCL). In some embodiments, the B-cell malignancy is a relapsed or refractory B-cell malignancy. In some embodiments, the B-cell malignancy is a BTK inhibitor-resistant B cell malignancy. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib suitable for oral administration. In some embodiments, ibrutinib and the BET inhibitor are administered simultaneously, sequentially or intermittently. In some embodiments, the combination further comprises a third therapeutic agent. In some embodiments, the third therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, are uses of a therapeutically effective amount of a combination comprising a BTK inhibitor and a BET inhibitor for treating a BTK inhibitor-resistant B cell malignancy comprising administering to a subject in need thereof a therapeutically effective amount of a combination comprising a BTK inhibitor and a BET inhibitor. In some embodiments, the combination provides a synergistic therapeutic effect compared to administration of the BTK inhibitor or the BET inhibitor alone. In some embodiments, the combination sensitizes a B-cell malignancy to the BTK inhibitor. In some embodiments, the BET inhibitor comprises CPI-0610, DUAL946, GSK525762, I-BET151, JQ1, OTX015, PFI-1, RVX-208, RVX2135, TEN-010, or a combination thereof. In some embodiments, the BET inhibitor is I-BET151, JQ1, OTX015, or a combination thereof. In some embodiments, the BET inhibitor is I-BET151. In some embodiments, the BET inhibitor is JQ1. In some embodiments, the BET inhibitor is OTX015. In some embodiments, the BTK inhibitor is a compound of Formula (A) or Formula (A1). In some embodiments, the BTK inhibitor is ibrutinib. In some embodiments, the BTK inhibitor is ibrutinib and the BET inhibitor is selected from among CPI-0610, DUAL946, GSK525762, I-BET151, JQ1, OTX015, PFI-1, RVX-208, RVX2135, TEN-010, or a combination thereof. In some embodiments, the BTK inhibitor is ibrutinib and the BET inhibitor is I-BET151, JQ1, OTX015, or a combination thereof. In some embodiments, the BTK inhibitor is ibrutinib and the BET inhibitor is I-BET151. In some embodiments, the BTK inhibitor is ibrutinib and the BET inhibitor is JQ1. In some embodiments, the BTK inhibitor is ibrutinib and the BET inhibitor is OTX015. In some embodiments, the B-cell malignancy is acute lymphoblastic leukemia (ALL), acute myelogenous leukemia (AML), chronic myelogenous leukemia (CML), acute monocytic leukemia (AMoL), chronic lymphocytic leukemia (CLL), high-risk CLL, small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is diffuse large B-cell lymphoma (DLBCL). In some embodiments, the DLBCL is activated B-cell diffuse large B-cell lymphoma (ABC-DLBCL). In some embodiments, the B-cell malignancy is a relapsed or refractory B-cell malignancy. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is suitable for oral administration. In some embodiments, ibrutinib and the BET inhibitor are administered simultaneously, sequentially or intermittently. In some embodiments, the combination further comprises a third therapeutic agent. In some embodiments, the third therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, are uses of a therapeutically effective amount of a combination comprising a BTK inhibitor and a BET inhibitor for treating a diffuse large B-cell lymphoma (DLBCL) comprising administering to a subject in need thereof a therapeutically effective amount of a combination comprising a BTK inhibitor and a BET inhibitor. In some embodiments, the combination provides a synergistic therapeutic effect compared to administration of the BTK inhibitor or the BET inhibitor alone. In some embodiments, the combination sensitizes a B-cell malignancy to the BTK inhibitor. In some embodiments, the BET inhibitor comprises CPI-0610, DUAL946, GSK525762, I-BET151, JQ1, OTX015, PFI-1, RVX-208, RVX2135, TEN-010, or a combination thereof. In some embodiments, the BET inhibitor is I-BET151, JQ1, OTX015, or a combination thereof. In some embodiments, the BET inhibitor is I-BET151. In some embodiments, the BET inhibitor is JQ1. In some embodiments, the BET inhibitor is OTX015. In some embodiments, the BTK inhibitor is a compound of Formula (A) or Formula (A1). In some embodiments, the BTK inhibitor is ibrutinib. In some embodiments, the BTK inhibitor is ibrutinib and the BET inhibitor is selected from among CPI-0610, DUAL946, GSK525762, I-BET151, JQ1, OTX015, PFI-1, RVX-208, RVX2135, TEN-010, or a combination thereof. In some embodiments, the BTK inhibitor is ibrutinib and the BET inhibitor is I-BET151, JQ1, OTX015, or a combination thereof. In some embodiments, the BTK inhibitor is ibrutinib and the BET inhibitor is I-BET151. In some embodiments, the BTK inhibitor is ibrutinib and the BET inhibitor is JQ1. In some embodiments, the BTK inhibitor is ibrutinib and the BET inhibitor is OTX015. In some embodiments, the DLBCL is activated B-cell diffuse large B-cell lymphoma (ABC-DLBCL). In some embodiments, the DLBCL is a relapsed or refractory DLBCL. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, ibrutinib and the BET inhibitor are administered simultaneously, sequentially or intermittently. In some embodiments, the method further comprises administering a third therapeutic agent. In some embodiments, the third therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, are uses of a therapeutically effective amount of a combination comprising a BTK inhibitor and a BET inhibitor for treating a B-cell malignancy associated with an elevated expression of c-MYC in an individual having an elevated expression level of c-MYC. In some embodiments, the elevated level of c-MYC is 1-fold, 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher compared to the expression level of the reference. In some embodiments, the reference level is the expression level of c-MYC in an individual who does not have a B-cell malignancy. In some embodiments, the combination provides a synergistic therapeutic effect compared to administration of the BTK inhibitor or the BET inhibitor alone. In some embodiments, the combination sensitizes a B-cell malignancy to the BTK inhibitor. In some embodiments, the BET inhibitor comprises CPI-0610, DUAL946, GSK525762, I-BET151, JQ1, OTX015, PFI-1, RVX-208, RVX2135, TEN-010, or a combination thereof. In some embodiments, the BET inhibitor is I-BET151, JQ1, OTX015, or a combination thereof. In some embodiments, the BET inhibitor is I-BET151. In some embodiments, the BET inhibitor is JQ1. In some embodiments, the BET inhibitor is OTX015. In some embodiments, the BTK inhibitor is a compound of Formula (A) or Formula (A1). In some embodiments, the BTK inhibitor is ibrutinib. In some embodiments, the BTK inhibitor is ibrutinib and the BET inhibitor is selected from among CPI-0610, DUAL946, GSK525762, I-BET151, JQ1, OTX015, PFI-1, RVX-208, RVX2135, TEN-010, or a combination thereof. In some embodiments, the BTK inhibitor is ibrutinib and the BET inhibitor is I-BET151, JQ1, OTX015, or a combination thereof. In some embodiments, the BTK inhibitor is ibrutinib and the BET inhibitor is I-BET151. In some embodiments, the BTK inhibitor is ibrutinib and the BET inhibitor is JQ1. In some embodiments, the BTK inhibitor is ibrutinib and the BET inhibitor is OTX015. In some embodiments, the B-cell malignancy is acute lymphoblastic leukemia (ALL), acute myelogenous leukemia (AML), chronic myelogenous leukemia (CML), acute monocytic leukemia (AMoL), chronic lymphocytic leukemia (CLL), high risk CLL, small lymphocytic lymphoma (SLL), high-risk SLL, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is diffuse large B-cell lymphoma (DLBCL). In some embodiments, the DLBCL is activated B-cell diffuse large B-cell lymphoma (ABC-DLBCL). In some embodiments, the B-cell malignancy is a relapsed or refractory B-cell malignancy. In some embodiments, the sample is a blood sample or a serum sample. In some embodiments, determining the expression level of c-MYC in the sample comprises measuring the amount of nucleic acid encoding c-MYC in the sample. In some embodiments, the sample comprises one or more tumor cells. In some embodiments, the nucleic acid is mRNA. In some embodiments, the method further comprises detecting the nucleic acid using a microarray. In some embodiments, the method further comprises amplification of the nucleic acid. In some embodiments, the amplification is a polymerase chain reaction. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered orally. In some embodiments, ibrutinib and the BET inhibitor are administered simultaneously, sequentially or intermittently. In some embodiments, the method further comprises administering a third therapeutic agent. In some embodiments, the third therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1F show cell growth curves illustrating BET inhibitors sensitized the ABC-DLBCL cell lines TMD8 and LY10 to ibrutinib.

FIG. 2 illustrates the interaction properties of ibrutinib in combination with BET inhibitors in various cell lines.

FIG. 3A shows the synergy score of the drug dose matrix data of a cell viability assay in TMD8 cells grown in the presence of a BET inhibitor (iBET151), ibrutinib, or a combination of the two drugs. The numbers in the plot indicate the percentage of growth inhibition of cells treated for 3 days with the corresponding compound combination relative to vehicle control-treated cells. FIG. 3B shows the corresponding isobologram, in which points to the left of the diagonal line represent synergistic combinations.

FIG. 3C shows the synergy score of the drug dose matrix data of a cell viability assay in TMD8 cells grown in the presence of a BET inhibitor (JQ1), ibrutinib, or a combination of the two drugs. The numbers in the plot indicate the percentage of growth inhibition of cells treated for 3 days with the corresponding compound combination relative to vehicle control-treated cells. FIG. 3D shows the corresponding isobologram, in which points to the left of the diagonal line represent synergistic combinations.

FIG. 3E shows the synergy score of the drug dose matrix data of a cell viability assay in TMD8 cells grown in the presence of a BET inhibitor (OTX015), ibrutinib, or a combination of the two drugs. The numbers in the plot indicate the percentage of growth inhibition of cells treated for 3 days with the corresponding compound combination relative to vehicle control-treated cells. FIG. 3F shows the corresponding isobologram, in which points to the left of the diagonal line represent synergistic combinations.

FIGS. 4A-4E illustrate that the combination of the BET inhibitor, JQ1, enhanced the growth suppression effect of ibrutinib on A20 tumors. Shown are plots of tumor size over time for individual animals treated with vehicle (FIG. 4B), ibrutinib FIG. 4C), JQ1 (FIG. 4D), or a combination of ibrutinib and JQ1 (FIG. 4E). FIG. 4A provides the average values and standard error for the corresponding individual data shown in FIGS. 4B-4E.

FIG. 5 illustrates that the combination of the BET inhibitor, JQ1, enhanced NK cytotoxicity in the A20 model.

### DETAILED DESCRIPTION OF THE INVENTION

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL,” means “about 5 μL” and also “5 μL.” Generally, the term “about” includes an amount that would be expected to be within experimental error.

The section headings used herein are for organizational purposes only and are not to be construed as limiting the subject matter described.

“Antibodies” and “immunoglobulins” (Igs) are glycoproteins having the same structural characteristics. The terms are used synonymously. In some instances the antigen specificity of the immunoglobulin may be known.

The term “antibody” is used in the broadest sense and covers fully assembled antibodies, antibody fragments that can bind antigen (e.g., Fab, F(ab′)₂, Fv, single chain antibodies, diabodies, antibody chimeras, hybrid antibodies, bispecific antibodies, humanized antibodies, and the like), and recombinant peptides comprising the forgoing.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a β-pleated-sheet configuration, connected by three CDRs, which form loops connecting, and in some cases forming part of, the β-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular toxicity, initiation of complement dependent cytotoxicity, and mast cell degranulation.

The term “hypervariable region,” when used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Antibody fragments” comprise a portion of an intact antibody, preferably the antigen-binding or variable region of the intact antibody. Examples of antibody fragments include Fab, Fab, F(ab′)2, and Fv fragments; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H1}) of the heavy chain. Fab fragments differ from Fab′ fragments by the addition of a few residues at the carboxy terminus of the heavy chain C_{H1} domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of human immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

### Bruton's Tyrosine Kinase (BTK) and BET Overview

BTK is a key regulator of B-cell development, activation, signaling, and survival (Kurosaki, Curr Op Imm, 2000, 276-281; Schaeffer and Schwartzberg, Curr Op Imm 2000, 282-288). It plays a role in a number of other hematopoietic cell signaling pathways, e.g., Toll like receptor (TLR) and cytokine receptor-mediated TNF-α production in macrophages, IgE receptor (FccRI) signaling in Mast cells, inhibition of Fas/APO-1 apoptotic signaling in B-lineage lymphoid cells, and collagen-stimulated platelet aggregation. See, e.g., C. A. Jeffries, et al., (2003), Journal of Biological Chemistry 278:26258-26264; N. J. Horwood, et al., (2003), The Journal of Experimental Medicine 197:1603-1611; Iwaki et al. (2005), Journal of Biological Chemistry 280(48):40261-40270; Vassilev et al. (1999), Journal of Biological Chemistry 274(3):1646-1656, and Quek et al. (1998), Current Biology 8(20):1137-1140.

Ibrutinib (PCI-32765) is an irreversible covalent inhibitor of BTK, inhibits proliferation, induces apoptosis, and has been shown to inhibit BTK in animal models. Further, clinical trials have demonstrated efficacy across several hematological malignancies (e.g. chronic lymphocytic leukemia (CLL) and diffuse large B-cell lymphoma (DLBCL)) including relapsed/refractory hematological malignancies. Indeed, about 70% of chronic lymphocytic leukemia (CLL) patient have demonstrated an objective complete or partial response in a clinical trial and an additional 15 to 20% of patients have a partial response with persistent lymphocytosis. At 26 months, the estimated progression-free survival rate among patients treated with ibrutinib is about 75%. For patients who have the activated B-cell like (ABC) subtype of DLBCL, the overall response rate is 41% and the overall survival is 9.7 month.

MYC is a transcription factor that participates in numerous cellular processes, such as cell proliferation, apoptosis, differentiation, metabolism and genome stability. Under certain cellular contexts, MYC can induce or suppress the expression of about 15% of all known genes. In addition, one study has shown that MYC is particularly enriched at the promoter regions of active genes, which functions to amplify existing transcriptional signals. The MYC gene includes three members, c-MYC, MYCN and MYCL. c-MYC is expressed ubiquitously in tissues and organs where as MYCN and MYCL are expressed predominately in the central nervous system and lung epithelium.

Under normal conditions, MYC basal expression level remains low. However, deregulated MYC resulted from translocation or genomic amplification leads to neoplastic transformation and tumor progression. Indeed, high levels of MYC expression are associated with a number of cancers, including non-Hodgkin lymphomas such as Burkitt's lymphoma, multiple myeloma, and DLBCL. Further, elevated expression level of MYC has been generally associated with poor prognosis and with aggressive malignancies including several types of lymphomas. For example, one study showed that elevated expression of MYC was present in about 80% of transformed large cell lymphomas, the aggressive form of large cell lymphoma. Further, several studies have shown that when MYC was silenced, tumor progression was also impaired, therefore, making MYC attractive as a therapeutic target. Inhibitors of the family of bromo and extra terminal (BET) proteins are known to downregulate MYC.

BET is a transcriptional regulator that is required for efficient expression of several growth promoting, anti-apoptotic genes, and cell cycle progression. BET family comprises BRD2, BRD3, BRD4 and BRDT. During transcription, the BET proteins are recruited to the chromatin via the N-terminal bromodomains (BRDs), in which this domain recognizes acetylated lysine residues in histone H3 and H4. Inhibitors of BET disrupt this BET-histone interaction and subsequently downregulates transcription of oncogenes including MYC.

MYC and BTK are important regulators of cellular processes and tumor progression. In light of the relationship between BET inhibition and MYC down-regulation and the relationship between overexpression of MYC and cancer, BET inhibitors are useful for treating MYC-associated diseases. Further, BET inhibitors in combination with BTK inhibitors such as ibrutinib are useful for treating hematological diseases.

Disclosed herein, in certain embodiments, are methods and compositions for treating a B-cell malignancy in a subject, comprising administering to the subject a therapeutically effective amount of a combination comprising a BTK inhibitor and a BET inhibitor. In some embodiments, disclosed herein are methods and compositions for treating a B-cell malignancy in a subject, comprising administering to the subject a therapeutically effective amount of a combination comprising ibrutinib and a BET inhibitor.

Further disclosed herein, in certain embodiments, are methods and compositions for treating a BTK inhibitor-resistant B cell malignancy, comprising administering to a subject in need thereof a therapeutically effective amount of a combination comprising a BTK inhibitor and a BET inhibitor. In some embodiments, disclosed herein are methods and compositions of treating a BTK inhibitor-resistant B cell malignancy, comprising administering to a subject in need thereof a therapeutically effective amount of a combination comprising ibrutinib and a BET inhibitor.

Also disclosed herein, in certain embodiments, are methods and compositions for treating a diffuse large B-cell lymphoma (DLBCL), comprising administering to a subject in need thereof a therapeutically effective amount of a combination comprising a BTK inhibitor and a BET inhibitor. In some embodiments, disclosed herein are methods and compositions of treating a diffuse large B-cell lymphoma (DLBCL), comprising administering to a subject in need thereof a therapeutically effective amount of a combination comprising ibrutinib and a BET inhibitor.

In certain embodiments, disclosed herein are methods and compositions for treating a B-cell malignancy or a TEC inhibitor-resistant B cell malignancy in a subject, comprising administering to the subject a therapeutically effective amount of a combination comprising a TEC inhibitor and a BET inhibitor. In certain embodiments, disclosed herein are methods and compositions for treating a B-cell malignancy or an ITK inhibitor-resistant B cell malignancy in a subject, comprising administering to the subject a therapeutically effective amount of a combination comprising an ITK inhibitor and a BET inhibitor.

### TEC Family Kinase Inhibitors

BTK is a member of the Tyrosine-protein kinase (TEC) family of kinases. In some embodiments, the TEC family comprises BTK, ITK, TEC, RLK and BMX. In some embodiments, a covalent TEC family kinase inhibitor inhibits the kinase activity of BTK, ITK, TEC, RLK and BMX. In some embodiments, a covalent TEC family kinase inhibitor is a BTK inhibitor. In some embodiments, a covalent TEC family kinase inhibitor is an ITK inhibitor. In some embodiments, a covalent TEC family kinase inhibitor is a TEC inhibitor. In some embodiments, a covalent TEC family kinase inhibitor is a RLK inhibitor. In some embodiments, a covalent TEC family kinase inhibitor is a BMK inhibitor.

### BTK Inhibitor Compounds Including Ibrutinib, and Pharmaceutically Acceptable Salts Thereof

The BTK inhibitor compound described herein (i.e. Ibrutinib) is selective for BTK and kinases having a cysteine residue in an amino acid sequence position of the tyrosine kinase that is homologous to the amino acid sequence position of cysteine 481 in BTK. The BTK inhibitor compound can form a covalent bond with Cys 481 of BTK (e.g., via a Michael reaction).

In some embodiments, the BTK inhibitor is a compound of Formula (A) having the structure:

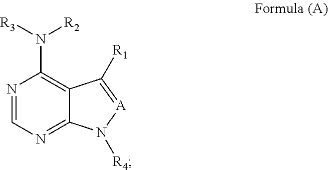

wherein:

A is N;

R₁ is phenyl-O-phenyl or phenyl-S-phenyl;

R₂ and R₃ are independently H;

R₄ is L₃-X-L₄-G, wherein,

L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;

X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;

L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;

or L₃, X and L₄ taken together form a nitrogen containing heterocyclic ring;

G is

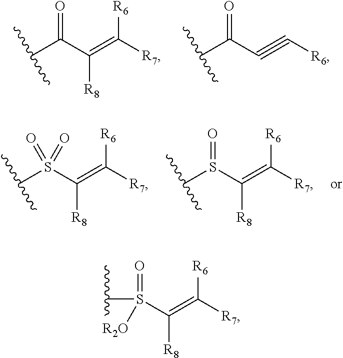

wherein,

R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;

each R₉ is independently selected from among H, substituted or unsubstituted lower alkyl, and substituted or unsubstituted lower cycloalkyl;

each R₁₀ is independently H, substituted or unsubstituted lower alkyl, or substituted or unsubstituted lower cycloalkyl; or

two R₁₀ groups can together form a 5-, 6-, 7-, or 8-membered heterocyclic ring; or

R₁₀ and R₁₁ can together form a 5-, 6-, 7-, or 8-membered heterocyclic ring; or each R₁₁ is independently selected from H or substituted or unsubstituted alkyl; or a pharmaceutically acceptable salt thereof. In some embodiments, L₃, X and L₄ taken together form a nitrogen containing heterocyclic ring. In some embodiments, the nitrogen containing heterocyclic ring is a piperidine group. In some embodiments, G is

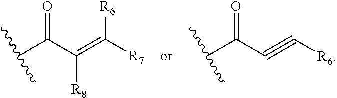

In some embodiments, the compound of Formula (A) is 1-[(3R)-3-[4-amino-3-(4-phenoxyphenyl)pyrazolo[3,4-d]pyrimidin-1-yl]piperidin-1-yl]prop-2-en-1-one.

In some embodiments, the BTK inhibitor is a compound having the structure of Formula (A1):

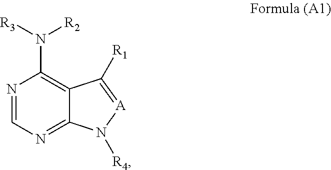

wherein
A is independently selected from N or CR₅;
R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkylene), or -(substituted or unsubstituted C₂-C₆ alkenylene);
R₂ and R₃ are independently selected from H, lower alkyl and substituted lower alkyl;
R₄ is L₃-X-L₄-G, wherein,
   L₃ is optional, and when present is a bond, or an optionally substituted group selected from alkylene, heteroalkylene, arylene, heteroarylene, alkylarylene, alkylheteroarylene, or alkylheterocycloalkylene;
   X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroarylene, arylene, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
   L₄ is optional, and when present is a bond, substituted or unsubstituted alkylene, substituted or unsubstituted cycloalkylene, substituted or unsubstituted alkenylene, substituted or unsubstituted alkynylene, substituted or unsubstituted arylene, substituted or unsubstituted heteroarylene, substituted or unsubstituted heterocyclene;
   or L₃, X and L₄ taken together form a nitrogen containing heterocyclic ring, or an optionally substituted group selected from alkyl, heteroalkyl, aryl, heteroaryl, alkylaryl, alkylheteroaryl, or alkylheterocycloalkyl;
   G is

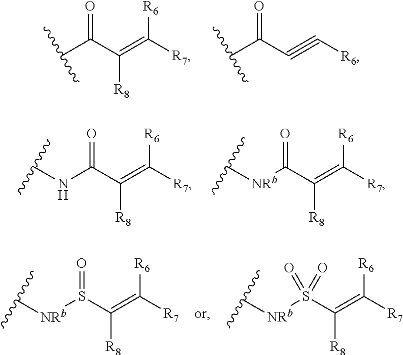

where R^{b} is H, substituted or unsubstituted alkyl, substituted or unsubstituted cycloalkyl; and either R₇ and R₈ are H;
   R₆ is H, substituted or unsubstituted C₁-C₄alkyl, substituted or unsubstituted C₁-C₄heteroalkyl, C₁-C₈alkylaminoalkyl, C₁-C₈hydroxyalkylaminoalkyl, C₁-C₈alkoxyalkylaminoalkyl, substituted or unsubstituted C₃-C₆cycloalkyl, substituted or unsubstituted C₁-C₈alkylC₃-C₆cycloalkyl, substituted or unsubstituted aryl, substituted or unsubstituted C₂-C₈heterocycloalkyl, substituted or unsubstituted heteroaryl, C₁-C₄alkyl(aryl), C₁-C₄alkyl(heteroaryl), C₁-C₈alkylethers, C₁-C₈alkylamides, or C₁-C₄alkyl(C₂-C₈heterocycloalkyl);
   R₆ and R₈ are H;
      R₇ is H, substituted or unsubstituted C₁-C₄alkyl, substituted or unsubstituted C₁-C₄heteroalkyl, C₁-C₈alkylaminoalkyl, C₁-C₈hydroxyalkylaminoalkyl, C₁-C₈alkoxyalkylaminoalkyl, substituted or unsubstituted C₃-C₆cycloalkyl, substituted or unsubstituted C₁-C₈alkylC₃-C₆cycloalkyl, substituted or unsubstituted aryl, substituted or unsubstituted C₂-C₈heterocycloalkyl, substituted or unsubstituted heteroaryl, C₁-C₄alkyl(aryl), C₁-C₄alkyl(heteroaryl), C₁-C₈alkylethers, C₁-C₈alkylamides, or C₁-C₄alkyl(C₂-C₈heterocycloalkyl); or
   R₇ and R₈ taken together form a bond;
      R₆ is H, substituted or unsubstituted C₁-C₄alkyl, substituted or unsubstituted C₁-C₄heteroalkyl, C₁-C₈alkylaminoalkyl, C₁-C₈hydroxyalkylaminoalkyl, C₁-C₈alkoxyalkylaminoalkyl, substituted or unsubstituted C₃-C₆cycloalkyl, substituted or unsubstituted C₁-C₈alkylC₃-C₆cycloalkyl, substituted or unsubstituted aryl, substituted or unsubstituted C₂-C₈heterocycloalkyl, substituted or unsubstituted heteroaryl, C₁-C₄alkyl(aryl), C₁-C₄alkyl(heteroaryl), C₁-C₈alkylethers, C₁-C₈alkylamides, or C₁-C₄alkyl(C₂-C₈heterocycloalkyl); or
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
R₉ is selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
R₁₁ is selected from H, —S(═O)₂R₈, —S(═O)₂NH₂, —C(O)R₈, —CN, —NO₂, heteroaryl, or heteroalkyl; and pharmaceutically active metabolites, pharmaceutically acceptable solvates, pharmaceutically acceptable salts, or pharmaceutically acceptable prodrugs thereof.

In some embodiments, A is independently selected from N. In some embodiments R₁ is L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkylene), or -(substituted or unsubstituted C₂-C₆ alkenylene). In a further embodiment, R₁ is L₂-(substituted or unsubstituted aryl) and L₂ is a bond. In a further embodiment, R₁ is L₂-(substituted aryl) wherein L₂ is a bond and aryl is substituted with L3-(substituted or unsubstitued heteroaryl) or L₃-(substituted or unsubstituted aryl). In a further embodiment, L₃ is a bond, O, S, NHC(O), C(O)NH.

In some embodiments, L₃, X and L₄ taken together form a nitrogen containing heterocyclic ring. In a further embodiment L₃, X and L₄ taken together form a pyrrolidine ring or a piperidine ring. In yet a further embodiment L₃, X and L₄ taken together form a piperidine ring. In some embodiments, G is

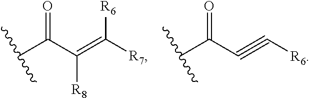

In some embodiments G is

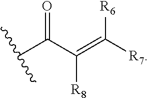

In some embodiments, R₆, R₇ and R₈ are H.

“Ibrutinib” or “1-((R)-3-(4-amino-3-(4-phenoxyphenyl)-1H-pyrazolo[3,4-d]pyrimidin-1-yl)piperidin-1-yl)prop-2-en-1-one” or “1-{(3R)-3-[4-amino-3-(4-phenoxyphenyl)-1H-pyrazolo[3,4-d]pyrimidin-1-yl]piperidin-1-yl}prop-2-en-1-one” or “2-Propen-1-one, 1-[(3R)-3-[4-amino-3-(4-phenoxyphenyl)-1H-pyrazolo[3,4-d]pyrimidin-1-yl]-1-piperidinyl-” or Ibrutinib or any other suitable name refers to the compound with the following structure:

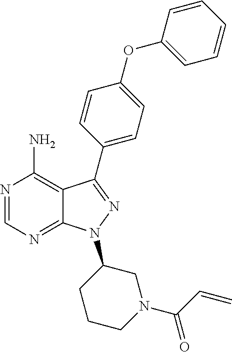 [SMILES: C=CC(=O)N1CCC[C@@H](n2nc(-c3ccc(Oc4ccccc4)cc3)c3c(N)ncnc32)C1]

A wide variety of pharmaceutically acceptable salts is formed from Ibrutinib and includes:
acid addition salts formed by reacting Ibrutinib with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like;
acid addition salts formed by reacting Ibrutinib with an inorganic acid, which includes hydrochloric acid, hydrobromic acid, sulfuric acid, nitric acid, phosphoric acid, hydroiodic acid, hydrofluoric acid, phosphorous acid, and the like.

The term “pharmaceutically acceptable salts” in reference to Ibrutinib refers to a salt of Ibrutinib, which does not cause significant irritation to a mammal to which it is administered and does not substantially abrogate the biological activity and properties of the compound.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol. In some embodiments, solvates of Ibrutinib, or pharmaceutically acceptable salts thereof, are conveniently prepared or formed during the processes described herein. In some embodiments, solvates of Ibrutinib are anhydrous. In some embodiments, Ibrutinib, or pharmaceutically acceptable salts thereof, exist in unsolvated form. In some embodiments, Ibrutinib, or pharmaceutically acceptable salts thereof, exist in unsolvated form and are anhydrous.

In yet other embodiments, Ibrutinib, or a pharmaceutically acceptable salt thereof, is prepared in various forms, including but not limited to, amorphous phase, crystalline forms, milled forms and nano-particulate forms. In some embodiments, Ibrutinib, or a pharmaceutically acceptable salt thereof, is amorphous. In some embodiments, Ibrutinib, or a pharmaceutically acceptable salt thereof, is amorphous and anhydrous. In some embodiments, Ibrutinib, or a pharmaceutically acceptable salt thereof, is crystalline. In some embodiments, Ibrutinib, or a pharmaceutically acceptable salt thereof, is crystalline and anhydrous.

In some embodiments, Ibrutinib is prepared as outlined in U.S. Pat. No. 7,514,444.

In some embodiments, the Btk inhibitor is ibrutinib (PCI-32765), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), ACP-196 (Acerta), PRN1008 (Principia), CTP-730 (Concert Pharmaceuticals), GDC-0853 (Genentech), or a combination thereof. In some embodiments, the BTK inhibitor is ibrutinib.

In some embodiments, the BTK inhibitor is 4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide (CGI-1746); 7-benzyl-1-(3-(piperidin-1-yl)propyl)-2-(4-(pyridin-4-yl)phenyl)-1H-imidazo[4,5-g]quinoxalin-6(5H)-one (CTA-056); (R)—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide (GDC-0834); 6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one (RN-486); N-[5-[5-(4-acetylpiperazine-1-carbonyl)-4-methoxy-2-methylphenyl]sulfanyl-1,3-thiazol-2-yl]-4-[(3,3-dimethylbutan-2-ylamino)methyl]benzamide (BMS-509744, HY-11092); or N-(5-((5-(4-Acetylpiperazine-1-carbonyl)-4-methoxy-2-methylphenyl)thio)thiazol-2-yl)-4-(((3-methylbutan-2-yl)amino)methyl)benzamide (HY11066); or a pharmaceutically acceptable salt thereof.

In some embodiments, the BTK inhibitor is:

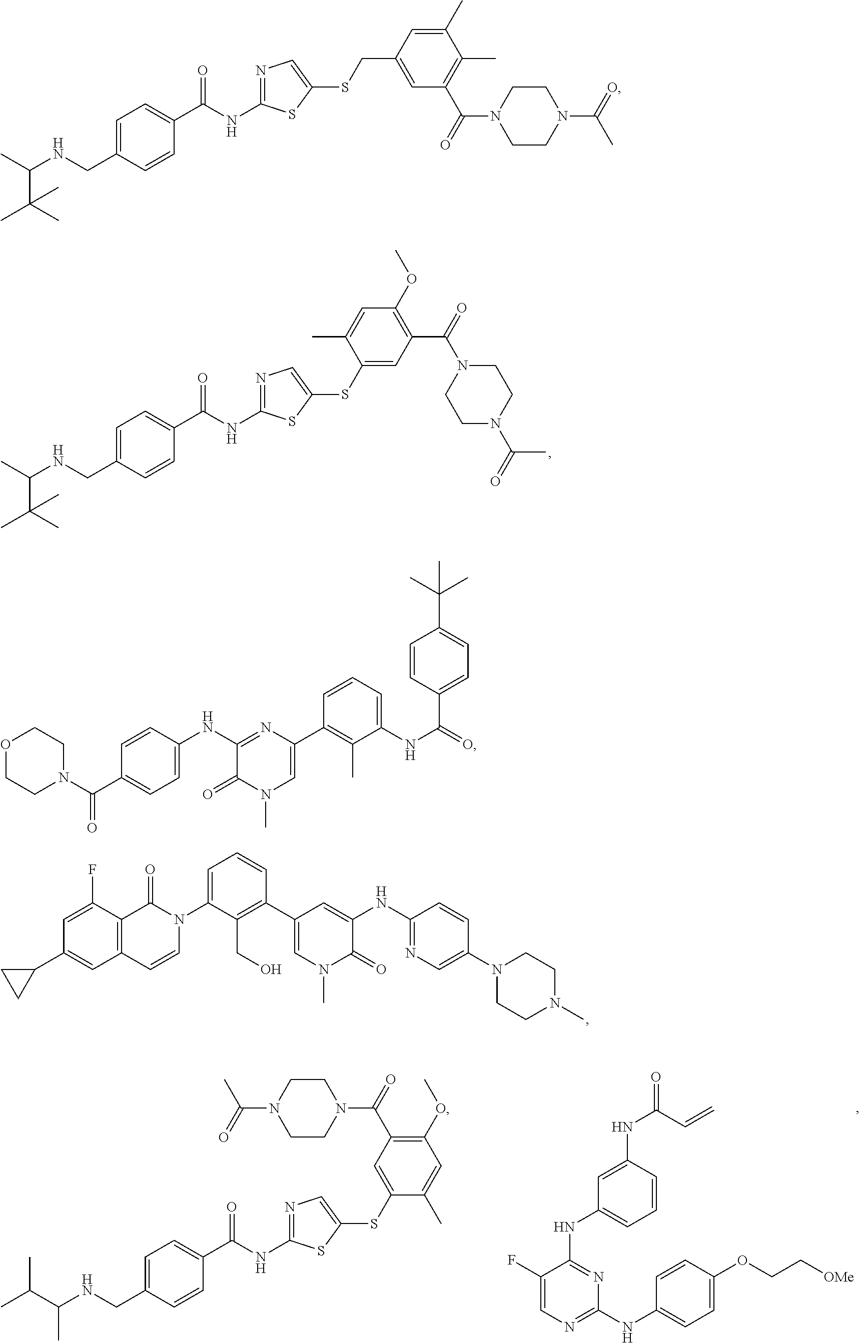

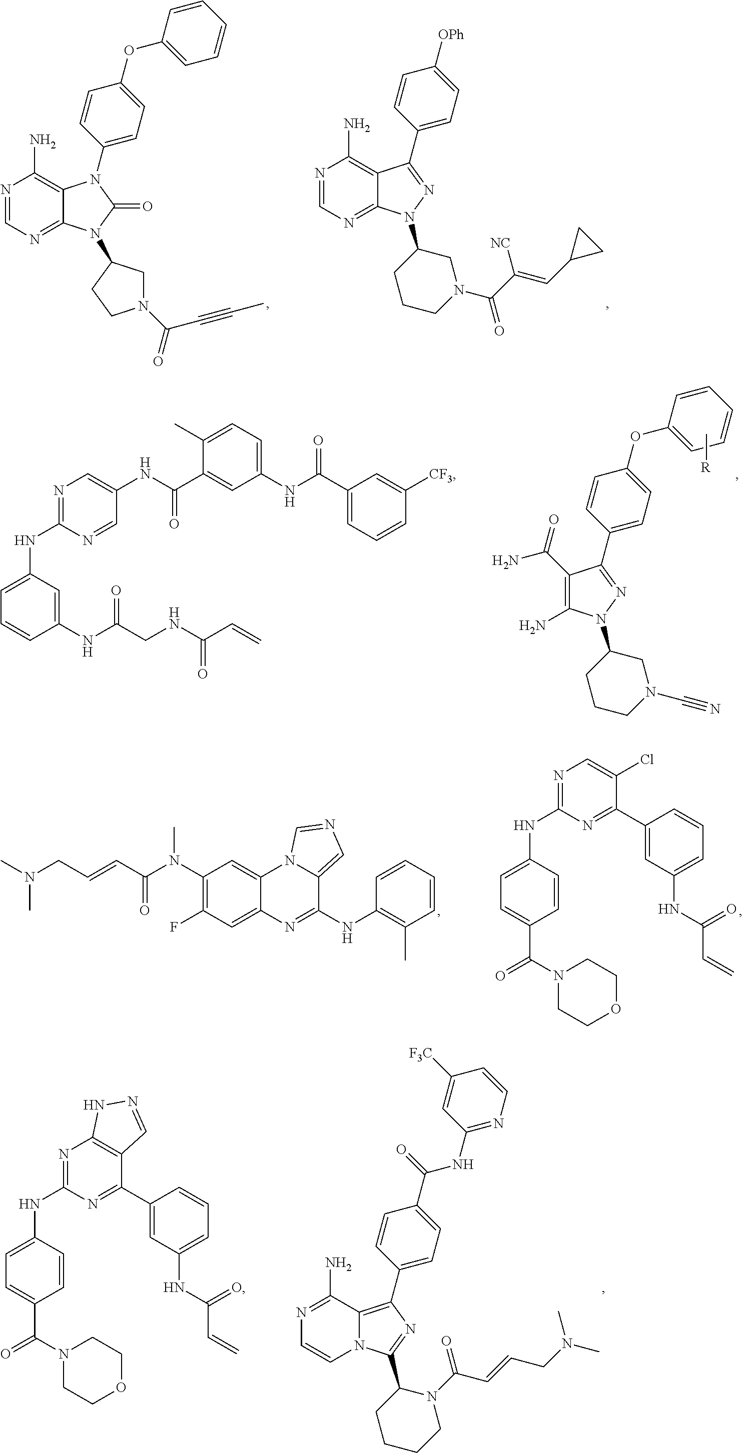

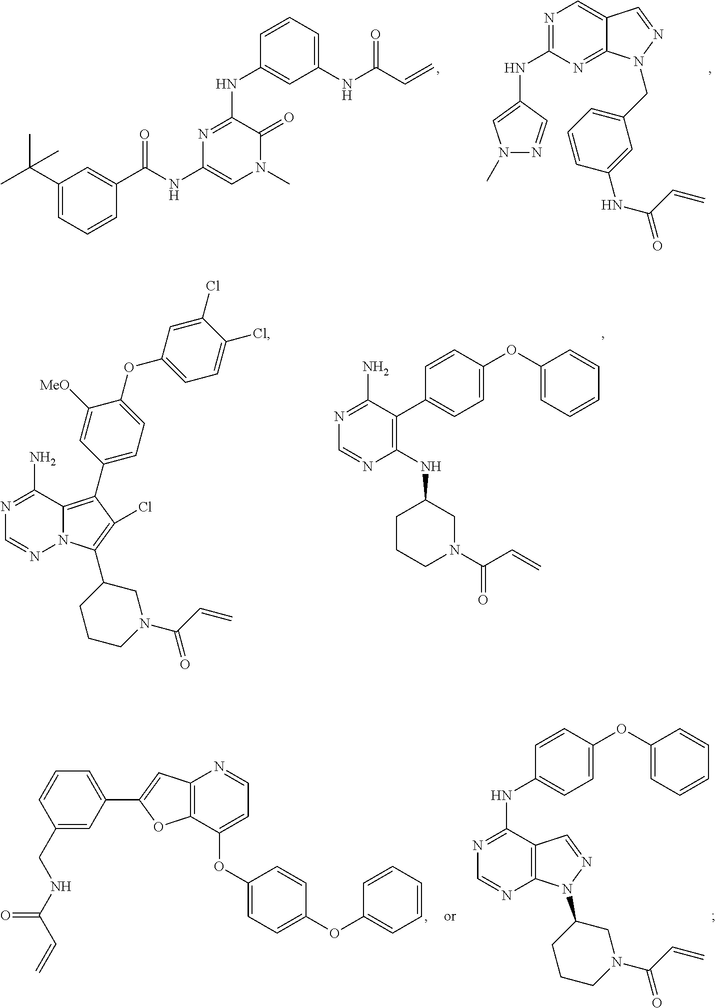 [SMILES: C=CC(=O)Nc1cccc(Nc2nc(NC(=O)c3cccc(C(C)(C)C)c3)cn(C)c2=O)c1 ; C=CC(=O)Nc1cccc(Cn2ncc3cnc(Nc4cnn(C)c4)nc32)c1 ; C=CC(=O)N1CCCC(c2c(Cl)c(-c3ccc(Oc4ccc(Cl)c(Cl)c4)c(OC)c3)c3c(N)ncnn23)C1 ; C=CC(=O)N1CCC[C@@H](Nc2ncnc(N)c2-c2ccc(Oc3ccccc3)cc2)C1 ; C=CC(=O)NCc1cccc(-c2cc3nccc(Oc4ccc(Oc5ccccc5)cc4)c3o2)c1 ; C=CC(=O)N1CCC[C@@H](n2ncc3c(Nc4ccc(Oc5ccccc5)cc4)ncnc32)C1]

or a pharmaceutically acceptable salt thereof.

### ITK Inhibitors

In some embodiments, the ITK inhibitor covalently binds to Cysteine 442 of ITK. In some embodiments, the ITK inhibitor is an ITK inhibitor compound described in W02002/0500071, which is incorporated by reference in its entirety. In some embodiments, the ITK inhibitor is an ITK inhibitor compound described in WO2005/070420, which is incorporated by reference in its entirety. In some embodiments, the ITK inhibitor is an ITK inhibitor compound described in WO2005/079791, which is incorporated by reference in its entirety. In some embodiments, the ITK inhibitor is an ITK inhibitor compound described in WO2007/076228, which is incorporated by reference in its entirety. In some embodiments, the ITK inhibitor is an ITK inhibitor compound described in WO2007/058832, which is incorporated by reference in its entirety. In some embodiments, the ITK inhibitor is an ITK inhibitor compound described in WO2004/016610, which is incorporated by reference in its entirety. In some embodiments, the ITK inhibitor is an ITK inhibitor compound described in WO2004/016611, which is incorporated by reference in its entirety. In some embodiments, the ITK inhibitor is an ITK inhibitor compound described in WO2004/016600, which is incorporated by reference in its entirety. In some embodiments, the ITK inhibitor is an ITK inhibitor compound described in WO2004/016615, which is incorporated by reference in its entirety. In some embodiments, the ITK inhibitor is an ITK inhibitor compound described in WO2005/026175, which is incorporated by reference in its entirety. In some embodiments, the ITK inhibitor is an ITK inhibitor compound described in WO2006/065946, which is incorporated by reference in its entirety. In some embodiments, the ITK inhibitor is an ITK inhibitor compound described in WO2007/027594, which is incorporated by reference in its entirety. In some embodiments, the ITK inhibitor is an ITK inhibitor compound described in WO2007/017455, which is incorporated by reference in its entirety. In some embodiments, the ITK inhibitor is an ITK inhibitor compound described in WO2008/025820, which is incorporated by reference in its entirety. In some embodiments, the ITK inhibitor is an ITK inhibitor compound described in WO2008/025821, which is incorporated by reference in its entirety. In some embodiments, the ITK inhibitor is an ITK inhibitor compound described in WO2008/025822, which is incorporated by reference in its entirety. In some embodiments, the ITK inhibitor is an ITK inhibitor compound described in WO2011/017219, which is incorporated by reference in its entirety. In some embodiments, the ITK inhibitor is an ITK inhibitor compound described in WO2011/090760, which is incorporated by reference in its entirety. In some embodiments, the ITK inhibitor is an ITK inhibitor compound described in WO2009/158571, which is incorporated by reference in its entirety. In some embodiments, the ITK inhibitor is an ITK inhibitor compound described in WO2009/051822, which is incorporated by reference in its entirety. In some embodiments, the ITK inhibitor is an ITK inhibitor compound described in U.S. Ser. No. 13/177,657, which is incorporated by reference in its entirety.

In some embodiments, the ITK inhibitor has a structure selected from:

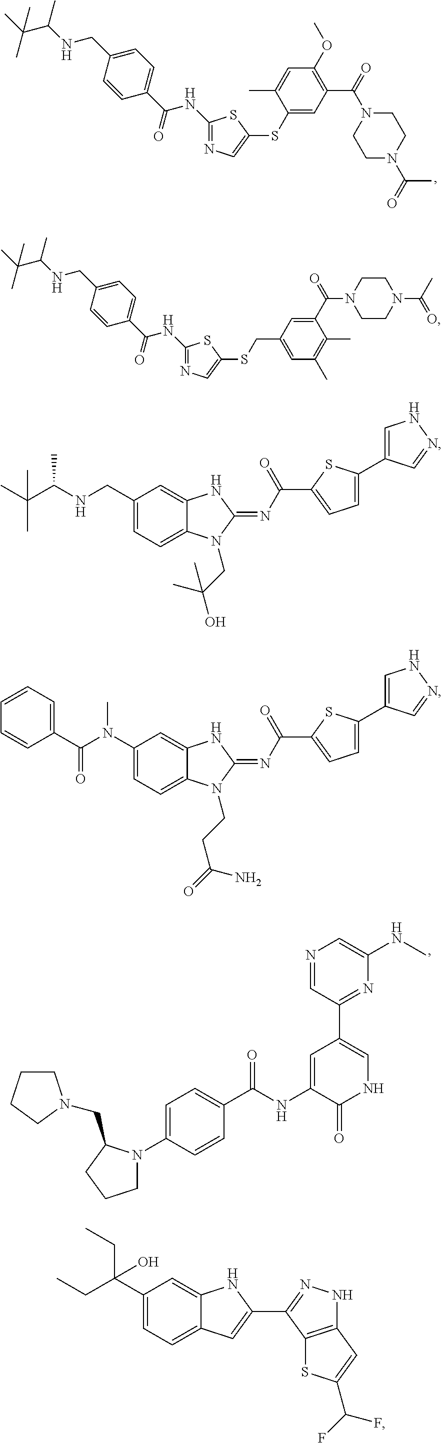

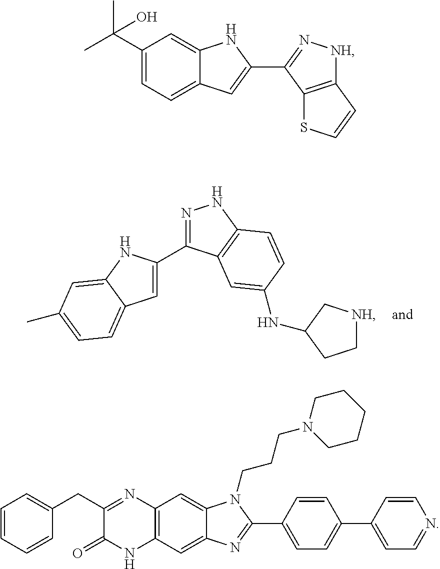

### BET Inhibitors

The BET inhibitors are small molecule compounds that target the members of the BET protein family, BRD2, BRD3, BRD4 and BRDT. In some embodiments, the BET inhibitors are pan-BET inhibitors that target BRD2, BRD3, BRD4 and/or BRDT. In some embodiments, the BET inhibitors are selective inhibitors that target BRD2, BRD3, BRD4 or BRDT. In some embodiments, the BET inhibitors include, but are not limited to, CPI-0610 (Constellation Pharmaceuticals), DUAL946, GSK525762 (I-BET762, GlaxoSmithKline), I-BET151 (GSK1210151), JQ1, OTX015 (OncoEthix SA), PFI-1 (PF-6405761, Pfizer), RVX-208 (Resverlogix), RVX2135 (Resverlogix), TEN-010 (Tensha Therapeutics, Inc), or a combination thereof. In some embodiments, the BET inhibitor is CPI-0610, DUAL946, GSK525762, I-BET151, JQ1, PFI-1, RVX-208, RVX2135, TEN-010, or a combination thereof. In some embodiments, the BET inhibitor is other than OTX015. DUAL946 is a dual BET/HDAC inhibitor which comprises a structural combination of I-BET295 and SAHA (see, Atkinson et al., “The structure based design of dual HDAC/BET inhibitors as novel epigenetic probes,” Med Chem Comm 5:342-351 (2014)). In some embodiments, the BET inhibitor is I-BET151, JQ1, OTX015, or a combination thereof. In some embodiments, the BET inhibitor is I-BET151. In some embodiments, the BET inhibitor is JQ1. In some embodiments, the BET inhibitor is OTX015.

In some embodiments, a BET inhibitor is a BET inhibitor disclosed in any of the following patent publications: WO2009084693; WO2012075383; WO2012075456; WO2012151512; WO2012068589; WO2014078257; WO2012143415; WO2012143413; WO2011054553; WO2011054845; WO2014001356; WO2014068402; WO2013064900; WO2013027168; US20140179648; US20140142102; and US20140140956.

### Hematological Malignancies

Hematological malignancies are a diverse group of cancer that affects the blood, bone marrow, and lymph nodes. In some embodiments, the hematological malignancy is a leukemia, a lymphoma, a myeloma, a non-Hodgkin's lymphoma, a Hodgkin's lymphoma, T-cell malignancy, or a B-cell malignancy.

In some embodiments, the hematological malignancy is a T-cell malignancy. In some embodiments, T-cell malignancies include peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some embodiments, the hematological malignancy is a B-cell malignancy. In some embodiments, B-cell malignancies include acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is diffuse large B-cell lymphoma (DLBCL). In some embodiments, the hematological malignancy is diffuse large B-cell lymphoma (DLBCL). In some embodiments, the DLBCL is an activated B-cell DLBCL (ABC-DLBCL), a germinal center B-cell like DLBCL (GBC-DLBCL), a double hit DLBCL (DH-DLBCL), or a triple hit DLBCL (TH-DLBCL).

In some embodiments, the hematological malignancy is a relapsed or refractory hematological malignancy. In some embodiments, the relapsed or refractory hematological malignancy is a relapsed or refractory T-cell malignancy. In some embodiments, the relapsed or refractory hematological malignancy is a relapsed or refractory B-cell malignancy. In some embodiments, the B-cell malignancy include acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the relapsed or refractory B-cell malignancy is diffuse large B-cell lymphoma (DLBCL). In some embodiments, the hematological malignancy is diffuse large B-cell lymphoma (DLBCL). In some embodiments, the DLBCL is an activated B-cell DLBCL (ABC-DLBCL), a germinal center B-cell like DLBCL (GBC-DLBCL), a double hit DLBCL (DH-DLBCL), or a triple hit DLBCL (TH-DLBCL). In some embodiments, the relapsed or refractory hematological malignancy is diffuse large B-cell lymphoma (DLBCL).

In some embodiments, the hematological malignancy is a relapsed hematological malignancy. In some embodiments, the hematological malignancy is a refractory hematological malignancy. In some embodiments, the refractory hematological malignancy contains an acquired resistance to a BTK inhibitor. In some embodiments, the BTK inhibitor is ibrutinib. In some embodiments, the refractory hematological malignancy is BTK-resistant hematological malignancy. In some embodiments, the hematological malignancy is BTK-resistant hematological malignancy.

### DLBCL

Disclosed herein, in certain embodiments, is a method for treating a diffuse large B-cell lymphoma (DLBCL) comprising administering to a subject in need thereof a therapeutically effective amount of a combination comprising a BTK inhibitor and a BET inhibitor. In certain embodiments, also disclosed herein, is a method for treating a diffuse large B-cell lymphoma (DLBCL) comprising administering to a subject in need thereof a therapeutically effective amount of a combination comprising ibrutinib and a BET inhibitor.

As used herein, the term “Diffuse large B-cell lymphoma (DLBCL)” refers to a neoplasm of the germinal center B lymphocytes with a diffuse growth pattern and a high-intermediate proliferation index. DLBCLs represent approximately 30% of all lymphomas and may present with several morphological variants including the centroblastic, immunoblastic, T-cell/histiocyte rich, anaplastic and plasmoblastic subtypes. Genetic tests have shown that there are different subtypes of DLBCL. These subtypes seem to have different outlooks (prognoses) and responses to treatment. DLBCL can affect any age group but occurs mostly in older people (the average age is mid-60s).

Disclosed herein, in certain embodiments, is a method for treating diffuse large B-cell lymphoma, activated B cell-like subtype (ABC-DLBCL) comprising administering to a subject in need thereof a therapeutically effective amount of a combination comprising a BTK inhibitor and a BET inhibitor. The ABC subtype of diffuse large B-cell lymphoma (ABC-DLBCL) is thought to arise from post germinal center B cells that are arrested during plasmatic differentiation. The ABC subtype of DLBCL (ABC-DLBCL) accounts for approximately 30% total DLBCL diagnoses. It is considered the least curable of the DLBCL molecular subtypes and, as such, patients diagnosed with the ABC-DLBCL typically display significantly reduced survival rates compared with individuals with other types of DLCBL. ABC-DLBCL is most commonly associated with chromosomal translocations deregulating the germinal center master regulator BCL6 and with mutations inactivating the PRDM1 gene, which encodes a transcriptional repressor required for plasma cell differentiation. In some embodiments, ABC-DLBCL contain mutations within the cytoplasmic tails of the B cell receptor subunits CD79A and CD79B.

In some embodiments, elevated expression of MYC is observed in DLBCL. In some embodiments, elevated expression of MYC is observed in the ABC subtypes of DLBCL. In some embodiments, disclosed herein is a method of treating MYC-driven DLBCL comprising administering to a subject in need thereof a therapeutically effective amount of a combination comprising a BTK inhibitor and a BET inhibitor. In some embodiments, disclosed herein is a method of treating MYC-driven ABC-DLBCL comprising administering to a subject in need thereof a therapeutically effective amount of a combination comprising a BTK inhibitor and a BET inhibitor.

### Diagnostic and Therapeutic Methods

### Biomarker

Disclosed herein, in certain embodiments, is a method of treating a B-cell malignancy associated with an elevated expression of c-MYC, comprising: (a) determining the expression level of c-MYC in a sample from an individual; and (b) administering to the individual a therapeutically effective amount of a combination comprising a BTK inhibitor and a BET inhibitor if the individual has an elevated expression level of c-MYC. Also disclosed herein, in certain embodiments, is a method of selecting an individual having a B-cell malignancy for therapy with a combination comprising a BTK inhibitor and a BET inhibitor, comprising: (a) measuring the expression level of c-MYC in a sample from the individual; (b) comparing the expression level of c-MYC with a reference level; and (c) characterizing the individual as a candidate for therapy with the combination comprising a BTK inhibitor and a BET inhibitor if the individual has an elevated level of c-MYC compared to the reference level. In some embodiments, ibrutinib is used in combination with a BET inhibitor. In some embodiments, an ITK inhibitor is used in combination with a BET inhibitor. In some embodiments, a TEC inhibitor is used in combination with a BET inhibitor.

In some embodiments, the level of expression of c-MYC in a sample is compared to the level of expression in a reference cell. In some embodiments, the reference cell is a cell that is non-cancerous. In some embodiments, the reference level is the expression level of c-MYC in an individual who does not have a B-cell malignancy.

In some embodiments, the elevated level of MYC is 1-fold, 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher compared to the expression of the reference level.

In some embodiments, the expression level of MYC is used to select an individual for treatment with the combination of a TEC inhibitor and a BET inhibitor. In some embodiments, an individual is administered a therapeutically effective combination of a TEC inhibitor and a BET inhibitor if the individual has an elevated expression of MYC. In some embodiments, an individual is not administered a therapeutically effective combination of a TEC inhibitor and a BET inhibitor if the individual does not have an elevated expression of MYC. In some embodiments, the TEC inhibitor is an inhibitor of BTK, ITK, TEC, RLK, or BMX. In some embodiments, the TEC inhibitor is an inhibitor of ITK. In some embodiment, the TEC inhibitor is an inhibitor of BTK.

In some embodiments, the expression level of MYC is used to select an individual for treatment with the combination of an ITK inhibitor and a BET inhibitor. In some embodiments, an individual is administered a therapeutically effective combination of an ITK inhibitor and a BET inhibitor if the individual has an elevated expression of MYC. In some embodiments, an individual is not administered a therapeutically effective combination of an ITK inhibitor and a BET inhibitor if the individual does not have an elevated expression of MYC. In some embodiments, the ITK inhibitor is an irreversible ITK inhibitor. In some embodiments, the ITK inhibitor is a reversible ITK inhibitor. In some embodiments, the BET inhibitor is CPI-0610, DUAL946, GSK525762, I-BET151, JQ1, OTX015, PFI-1, RVX-208, RVX2135, or TEN-010.

In some embodiments, the expression level of MYC is used to select an individual for treatment with the combination of a BTK inhibitor and a BET inhibitor. In some embodiments, an individual is administered a therapeutically effective combination of a BTK inhibitor and a BET inhibitor if the individual has an elevated expression of MYC. In some embodiments, an individual is not administered a therapeutically effective combination of a BTK inhibitor and a BET inhibitor if the individual does not have an elevated expression of MYC. In some embodiments, the BTK inhibitor is an irreversible BTK inhibitor. In some embodiments, the BTK inhibitor is a reversible BTK inhibitor. In some embodiments, the BTK inhibitor is ibrutinib (PCI-32765), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), ACP-196 (Acerta), PRN1008 (Principia), CTP-730 (Concert Pharmaceuticals), GDC-0853 (Genentech), or a combination thereof. In some embodiments, the BTK inhibitor is ibrutinib. In some embodiments, the BET inhibitor is CPI-0610, DUAL946, GSK525762, I-BET151, JQ1, OTX015, PFI-1, RVX-208, RVX2135, or TEN-010.

In some embodiments, the expression level of MYC is used to select an individual for treatment with the combination of ibrutinib and a BET inhibitor. In some embodiments, an individual is administered a therapeutically effective combination of ibrutinib and a BET inhibitor if the individual has an elevated expression of MYC. In some embodiments, an individual is not administered a therapeutically effective combination of ibrutinib and a BET inhibitor if the individual does not have an elevated expression of MYC. In some embodiments, the BET inhibitor is CPI-0610, DUAL946, GSK525762, I-BET151, JQ1, OTX015, PFI-1, RVX-208, RVX2135, or TEN-010.

In some embodiments, the expression level of MYC is used to assess or monitor the efficacy of the treatment with the combination of a TEC inhibitor and a BET inhibitor. In some embodiments, the treatment with the combination of a TEC inhibitor and a BET inhibitor is continued if the elevated level of MYC persists. In some embodiments, the treatment with the combination of a TEC inhibitor and a BET inhibitor is discontinued if the elevated level of MYC is decreased to near reference level. In some embodiments, the TEC inhibitor is an inhibitor of BTK, ITK, TEC, RLK, or BMX. In some embodiments, the TEC inhibitor is an inhibitor of ITK. In some embodiment, the TEC inhibitor is an inhibitor of BTK.

In some embodiments, the expression level of MYC is used to assess or monitor the efficacy of the treatment with the combination of a BTK inhibitor and a BET inhibitor. In some embodiments, the treatment with the combination of a BTK inhibitor and a BET inhibitor is continued if the elevated level of MYC persists. In some embodiments, the treatment with the combination of a BTK inhibitor and a BET inhibitor is discontinued if the elevated level of MYC is decreased to near reference level. In some embodiments, the ITK inhibitor is an irreversible ITK inhibitor. In some embodiments, the ITK inhibitor is a reversible ITK inhibitor. In some embodiments, the BET inhibitor is CPI-0610, DUAL946, GSK525762, I-BET151, JQ1, OTX015, PFI-1, RVX-208, RVX2135, or TEN-010.

In some embodiments, the expression level of MYC is used to assess or monitor the efficacy of the treatment with the combination of a BTK inhibitor and a BET inhibitor. In some embodiments, the treatment with the combination of a BTK inhibitor and a BET inhibitor is continued if the elevated level of MYC persists. In some embodiments, the treatment with the combination of a BTK inhibitor and a BET inhibitor is discontinued if the elevated level of MYC is decreased to near reference level. In some embodiments, the BTK inhibitor is an irreversible BTK inhibitor. In some embodiments, the BTK inhibitor is a reversible BTK inhibitor. In some embodiments, the BTK inhibitor is ibrutinib (PCI-32765), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), ACP-196 (Acerta), PRN1008 (Principia), CTP-730 (Concert Pharmaceuticals), GDC-0853 (Genentech), or a combination thereof. In some embodiments, the BTK inhibitor is ibrutinib. In some embodiments, the BET inhibitor is CPI-0610, DUAL946, GSK525762, I-BET151, JQ1, OTX015, PFI-1, RVX-208, RVX2135, or TEN-010.

In some embodiments, the expression level of MYC is used to assess or monitor the efficacy of the treatment with the combination of ibrutinib and a BET inhibitor. In some embodiments, the treatment with the combination of ibrutinib and a BET inhibitor is continued if the elevated level of MYC persists. In some embodiments, the treatment with the combination of ibrutinib and a BET inhibitor is discontinued if the elevated level of MYC is decreased to near reference level. In some embodiments, the BET inhibitor is CPI-0610, DUAL946, GSK525762, I-BET151, JQ1, OTX015, PFI-1, RVX-208, RVX2135, or TEN-010.

### Diagnostic Methods

Methods for determining the expression or presence of biomarkers such as MYC are well known in the art. Circulating levels of biomarkers in a blood sample obtained from a candidate subject are measured, for example, by ELISA, radioimmunoassay (RIA), electrochemiluminescence (ECL), Western blot, multiplexing technologies, or other similar methods. Cell surface expression of biomarkers are measured, for example, by flow cytometry, immunohistochemistry, Western Blot, immunoprecipitation, magnetic bead selection, and quantification of cells expressing either of these cell surface markers. Biomarker RNA expression levels could be measured by RT-PCR, Qt-PCR, microarray, Northern blot, or other similar technologies.

As disclosed herein, determining the expression or presence of the biomarker of interest at the protein or nucleotide level are accomplished using any detection method known to those of skill in the art. By “detecting expression” or “detecting the level of is intended determining the expression level or presence of a biomarker protein or gene in the biological sample. Thus, “detecting expression” encompasses instances where a biomarker is determined not to be expressed, not to be detectably expressed, expressed at a low level, expressed at a normal level, or overexpressed.

In certain aspects of the method provided herein, the one or more subpopulation of lymphocytes are isolated, detected or measured. In certain embodiments, the one or more subpopulation of lymphocytes are isolated, detected or measured using immunophenotyping techniques. In other embodiments, the one or more subpopulation of lymphocytes are isolated, detected or measured using fluorescence activated cell sorting (FACS) techniques.

In certain aspects, the expression or presence of these various biomarkers and any clinically useful prognostic markers in a biological sample are detected at the protein or nucleic acid level, using, for example, immunohistochemistry techniques or nucleic acid-based techniques such as in situ hybridization and RT-PCR. In one embodiments, the expression or presence of one or more biomarkers is carried out by a means for nucleic acid amplification, a means for nucleic acid sequencing, a means utilizing a nucleic acid microarray (DNA and RNA), or a means for in situ hybridization using specifically labeled probes.

In other embodiments, the determining the expression or presence of one or more biomarkers is carried out through gel electrophoresis. In one embodiment, the determination is carried out through transfer to a membrane and hybridization with a specific probe.

In other embodiments, the determining the expression or presence of one or more biomarkers carried out by a diagnostic imaging technique.

In still other embodiments, the determining the expression or presence of one or more biomarkers carried out by a detectable solid substrate. In one embodiment, the detectable solid substrate is paramagnetic nanoparticles functionalized with antibodies.

In another aspect, provided herein are methods for detecting or measuring residual lymphoma following a course of treatment in order to guide continuing or discontinuing treatment or changing from one therapeutic regimen to another comprising determining the expression or presence of one or more biomarkers from one or more subpopulation of lymphocytes in a subject wherein the course of treatment is treatment with a Btk inhibitor (e.g., ibrutinib).

Methods for detecting expression of the biomarkers described herein, within the test and control biological samples comprise any methods that determine the quantity or the presence of these markers either at the nucleic acid or protein level. Such methods are well known in the art and include but are not limited to western blots, northern blots, ELISA, immunoprecipitation, immunofluorescence, flow cytometry, immunohistochemistry, nucleic acid hybridization techniques, nucleic acid reverse transcription methods, and nucleic acid amplification methods. In particular embodiments, expression of a biomarker is detected on a protein level using, for example, antibodies that are directed against specific biomarker proteins. These antibodies are used in various methods such as Western blot, ELISA, multiplexing technologies, immunoprecipitation, or immunohistochemistry techniques. In some embodiments, detection of biomarkers is accomplished by ELISA. In some embodiments, detection of biomarkers is accomplished by electrochemiluminescence (ECL).

Any means for specifically identifying and quantifying a biomarker (for example, biomarker, a biomarker of cell survival or proliferation, a biomarker of apoptosis, a biomarker of a Btk-mediated signaling pathway) in the biological sample of a candidate subject is contemplated. Thus, in some embodiments, expression level of a biomarker protein of interest in a biological sample is detected by means of a binding protein capable of interacting specifically with that biomarker protein or a biologically active variant thereof. In some embodiments, labeled antibodies, binding portions thereof, or other binding partners are used. The word “label” when used herein refers to a detectable compound or composition that is conjugated directly or indirectly to the antibody so as to generate a “labeled” antibody. In some embodiments, the label is detectable by itself (e.g., radioisotope labels or fluorescent labels) or, in the case of an enzymatic label, catalyzes chemical alteration of a substrate compound or composition that is detectable.

The antibodies for detection of a biomarker protein are either monoclonal or polyclonal in origin, or are synthetically or recombinantly produced. The amount of complexed protein, for example, the amount of biomarker protein associated with the binding protein, for example, an antibody that specifically binds to the biomarker protein, is determined using standard protein detection methodologies known to those of skill in the art. A detailed review of immunological assay design, theory and protocols are found in numerous texts in the art (see, for example, Ausubel et al., eds. (1995) Current Protocols in Molecular Biology) (Greene Publishing and Wiley-Interscience, NY)); Coligan et al., eds. (1994) Current Protocols in Immunology (John Wiley & Sons, Inc., New York, N.Y.).

The choice of marker used to label the antibodies will vary depending upon the application. However, the choice of the marker is readily determinable to one skilled in the art. These labeled antibodies are used in immunoassays as well as in histological applications to detect the presence of any biomarker or protein of interest. The labeled antibodies are either polyclonal or monoclonal. Further, the antibodies for use in detecting a protein of interest are labeled with a radioactive atom, an enzyme, a chromophoric or fluorescent moiety, or a colorimetric tag as described elsewhere herein. The choice of tagging label also will depend on the detection limitations desired. Enzyme assays (ELISAs) typically allow detection of a colored product formed by interaction of the enzyme-tagged complex with an enzyme substrate. Radionuclides that serve as detectable labels include, for example, 1-131, 1-123, 1-125, Y-90, Re-188, Re-186, At-211, Cu-67, Bi-212, and Pd-109. Examples of enzymes that serve as detectable labels include, but are not limited to, horseradish peroxidase, alkaline phosphatase, beta-galactosidase, and glucose-6-phosphate dehydrogenase. Chromophoric moieties include, but are not limited to, fluorescein and rhodamine. The antibodies are conjugated to these labels by methods known in the art. For example, enzymes and chromophoric molecules are conjugated to the antibodies by means of coupling agents, such as dialdehydes, carbodiimides, dimaleimides, and the like. Alternatively, conjugation occurs through a ligand-receptor pair. Examples of suitable ligand-receptor pairs are biotin-avidin or biotin-streptavidin, and antibody-antigen.

In certain embodiments, expression or presence of one or more biomarkers or other proteins of interest within a biological sample, for example, a sample of bodily fluid, is determined by radioimmunoassays or enzyme-linked immunoassays (ELISAs), competitive binding enzyme-linked immunoassays, dot blot (see, for example, Promega Protocols and Applications Guide, Promega Corporation (1991), Western blot (see, for example, Sambrook et al. (1989) Molecular Cloning, A Laboratory Manual, Vol. 3, Chapter 18 (Cold Spring Harbor Laboratory Press, Plainview, N.Y.), chromatography such as high performance liquid chromatography (HPLC), or other assays known in the art. Thus, the detection assays involve steps such as, but not limited to, immunoblotting, immunodiffusion, immunoelectrophoresis, or immunoprecipitation.

In certain other embodiments, the methods of the invention are useful for identifying and treating cancer, including those listed above, that are refractory to (i.e., resistant to, or have become resistant to) first-line oncotherapeutic treatments.

In some embodiments, the expression or presence of one or more of the biomarkers described herein are also determined at the nucleic acid level. Nucleic acid-based techniques for assessing expression are well known in the art and include, for example, determining the level of biomarker mRNA in a biological sample. Many expression detection methods use isolated RNA. Any RNA isolation technique that does not select against the isolation of mRNA is utilized for the purification of RNA (see, e.g., Ausubel et al., ed. (1987-1999) Current Protocols in Molecular Biology (John Wiley & Sons, New York). Additionally, large numbers of tissue samples are readily processed using techniques well known to those of skill in the art, such as, for example, the single-step RNA isolation process disclosed in U.S. Pat. No. 4,843,155.

Thus, in some embodiments, the detection of a biomarker or other protein of interest is assayed at the nucleic acid level using nucleic acid probes. The term “nucleic acid probe” refers to any molecule that is capable of selectively binding to a specifically intended target nucleic acid molecule, for example, a nucleotide transcript. Probes are synthesized by one of skill in the art, or derived from appropriate biological preparations. Probes are specifically designed to be labeled, for example, with a radioactive label, a fluorescent label, an enzyme, a chemiluminescent tag, a colorimetric tag, or other labels or tags that are discussed above or that are known in the art. Examples of molecules that are utilized as probes include, but are not limited to, RNA and DNA.

For example, isolated mRNA are used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that hybridize to the mRNA encoded by the gene being detected. The nucleic acid probe comprises of, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker, biomarker described herein above. Hybridization of an mRNA with the probe indicates that the biomarker or other target protein of interest is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in a gene chip array. A skilled artisan readily adapts known mRNA detection methods for use in detecting the level of mRNA encoding the biomarkers or other proteins of interest.

An alternative method for determining the level of an mRNA of interest in a sample involves the process of nucleic acid amplification, e.g., by RT-PCR (see, for example, U.S. Pat. No. 4,683,202), ligase chain reaction (Barany (1991) Proc. Natl. Acad. Sci. USA 88:189 193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan0 System).

Expression levels of an RNA of interest are monitored using a membrane blot (such as used in hybridization analysis such as Northern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of expression also comprises using nucleic acid probes in solution.

In one embodiment of the invention, microarrays are used to determine expression or presence of one or more biomarkers. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261, incorporated herein by reference in its entirety. In some embodiments, an array is fabricated on a surface of virtually any shape or even a multiplicity of surfaces. In some embodiments, an array is a planar array surface. In some embodiments, arrays include peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. In some embodiments, arrays are packaged in such a manner as to allow for diagnostics or other manipulation of an all-inclusive device.

### Samples

In some embodiments, the sample for use in the methods is obtained from cells of a hematological malignant cell line. In some embodiments, the sample is obtained from cells of a acute lymphoblastic leukemia (ALL), acute myelogenous leukemia (AML), chronic myelogenous leukemia (CML), acute monocytic leukemia (AMoL), chronic lymphocytic leukemia (CLL), high risk CLL, small lymphocytic lymphoma (SLL), high risk SLL, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis cell line. In some embodiments, the sample is obtained from cells of a DLBCL cell line.

In some embodiments, the sample is a DLBCL cell or population of DLBCL cells. In some embodiments, the level of expression of c-MYC in a sample is compared to the level of expression in a reference DLBCL cell line. In some embodiments, the level of expression of c-MYC in a sample is compared to the level of expression in a reference DLBCL cell or population of DLBCL cells that is known to be resistant to treatment with a BTK inhibitor. In some embodiments, the level of expression of c-MYC in a sample is compared to the level of expression in a reference DLBCL cell or population of DLBCL cells that is known to be sensitive to treatment with a BTK inhibitor. In some embodiments, the level of expression of c-MYC in a sample is compared to the level of expression in a reference DLBCL cell line that is known to be resistant to treatment with a BTK inhibitor. In some embodiments, the level of expression of c-MYC in a sample is compared to the level of expression in a reference DLBCL cell line that is known to be sensitive to treatment with a BTK inhibitor. In some embodiments, the DLBCL cell line is an activated B-cell-like (ABC)-DLBCL cell line. In some embodiments, the DLBCL cell line is a germinal center B-cell-like (GCB)-DLBCL cell line. In some embodiments, the DLBCL cell line is OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, or TMD8. In some embodiments, the DLBCL cell line that is sensitive to treatment with a BTK inhibitor is TMD8, HBL-1 or OCI-Ly10. In some embodiments, the DLBCL cell line that is resistant to treatment with a BTK inhibitor is OCI-Ly3, DB or OCI-Ly19.

In some embodiments, the sample for use in the methods is from any tissue or fluid from a patient. Samples include, but are not limited, to whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In particular embodiments, the sample is a blood serum sample. In particular embodiments, the sample is from a fluid or tissue that is part of, or associated with, the lymphatic system or circulatory system. In some embodiments, the sample is a blood sample that is a venous, arterial, peripheral, tissue, cord blood sample. In particular embodiments, the sample is a blood cell sample containing one or more peripheral blood mononuclear cells (PBMCs). In some embodiments, the sample contains one or more circulating tumor cells (CTCs). In some embodiments, the sample contains one or more disseminated tumor cells (DTC, e.g., in a bone marrow aspirate sample).

In some embodiments, the samples are obtained from the individual by any suitable means of obtaining the sample using well-known and routine clinical methods. Procedures for obtaining fluid samples from an individual are well known. For example, procedures for drawing and processing whole blood and lymph are well-known and can be employed to obtain a sample for use in the methods provided. Typically, for collection of a blood sample, an anti-coagulation agent (e.g., EDTA, or citrate and heparin or CPD (citrate, phosphate, dextrose) or comparable substances) is added to the sample to prevent coagulation of the blood. In some examples, the blood sample is collected in a collection tube that contains an amount of EDTA to prevent coagulation of the blood sample.

In some embodiments, the collection of a sample from the individual is performed at regular intervals, such as, for example, one day, two days, three days, four days, five days, six days, one week, two weeks, weeks, four weeks, one month, two months, three months, four months, five months, six months, one year, daily, weekly, bimonthly, quarterly, biyearly or yearly.

In some embodiments, the collection of a sample is performed at a predetermined time or at regular intervals relative to treatment with a combination of a TEC inhibitor and a BET inhibitor. In some embodiments, the TEC inhibitor is a BTK inhibitor, an ITK inhibitor, a TEC inhibitor, a RLK inhibitor, or a BMX inhibitor. In some embodiments, the TEC inhibitor is an ITK inhibitor. In some embodiments, the TEC inhibitor is a BTK inhibitor.

In some embodiments, the collection of a sample is performed at a predetermined time or at regular intervals relative to treatment with a combination of an ITK inhibitor and a BET inhibitor. For example, a sample is collected from a patient at a predetermined time or at regular intervals prior to, during, or following treatment or between successive treatments with a combination of an ITK inhibitor and a BET inhibitor. In particular examples, a sample is obtained from a patient prior to administration of a combination of an ITK inhibitor and a BET inhibitor, and then again at regular intervals after treatment with the combination of the ITK inhibitor and the BET inhibitor has been effected. In some embodiments, the patient is administered a combination of an ITK inhibitor and a BET inhibitor and one or more additional therapeutic agents. In some embodiments, the ITK inhibitor is an irreversible ITK inhibitor. In some embodiments, the ITK inhibitor is a reversible ITK inhibitor. In some embodiments, the BET inhibitor is selected from among CPI-0610, DUAL946, GSK525762, I-BET151, JQ1, OTX015, PFI-1, RVX-208, RVX2135, TEN-010, and a combination thereof. In some embodiments, the BET inhibitor is I-BET151, JQ1, OTX015, or a combination thereof. In some embodiments, the BET inhibitor is I-BET151. In some embodiments, the BET inhibitor is JQ1. In some embodiments, the BET inhibitor is OTX015.

In some embodiments, the collection of a sample is performed at a predetermined time or at regular intervals relative to treatment with a combination of a BTK inhibitor and a BET inhibitor. For example, a sample is collected from a patient at a predetermined time or at regular intervals prior to, during, or following treatment or between successive treatments with a combination of a BTK inhibitor and a BET inhibitor. In particular examples, a sample is obtained from a patient prior to administration of a combination of a BTK inhibitor and a BET inhibitor, and then again at regular intervals after treatment with the combination of the BTK inhibitor and the BET inhibitor has been effected. In some embodiments, the patient is administered a combination of a BTK inhibitor and a BET inhibitor and one or more additional therapeutic agents. In some embodiments, the BTK inhibitor is an irreversible BTK inhibitor. In some embodiments, the BTK inhibitor is a reversible BTK inhibitor. In some embodiments, the BTK inhibitor is ibrutinib. In some embodiments, the BTK inhibitor is ibrutinib (PCI-32765), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK417891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), ACP-196 (Acerta), PRN1008 (Principia), CTP-730 (Concert Pharmaceuticals), GDC-0853 (Genentech), or a combination thereof. In some embodiments, the BET inhibitor is selected from among CPI-0610, DUAL946, GSK525762, I-BET151, JQ1, OTX015, PFI-1, RVX-208, RVX2135, TEN-010, or a combination thereof. In some embodiments, the BET inhibitor is I-BET151, JQ1, OTX015, or a combination thereof. In some embodiments, the BET inhibitor is I-BET151. In some embodiments, the BET inhibitor is JQ1. In some embodiments, the BET inhibitor is OTX015.

In some embodiments, the collection of a sample is performed at a predetermined time or at regular intervals relative to treatment with a combination of ibrutinib and a BET inhibitor. For example, a sample is collected from a patient at a predetermined time or at regular intervals prior to, during, or following treatment or between successive treatments with a combination of ibrutinib and a BET inhibitor. In particular examples, a sample is obtained from a patient prior to administration of a combination of ibrutinib and a BET inhibitor, and then again at regular intervals after treatment with the combination of ibrutinib and the BET inhibitor has been effected. In some embodiments, the patient is administered a combination of ibrutinib and a BET inhibitor and one or more additional therapeutic agents. In some embodiments, the BET inhibitor is selected from among CPI-0610, DUAL946, GSK525762, I-BET151, JQ1, OTX015, PFI-1, RVX-208, RVX2135, TEN-010, and a combination thereof.

### Additional Combination Therapies

In certain embodiments, a TEC inhibitor and a BET inhibitor are administered in combination with an additional therapeutic agent for the treatment of a hematological malignancy. In some embodiments, the TEC inhibitor is a BTK inhibitor, an ITK inhibitor, a TEC inhibitor, a RLK inhibitor, or a BMX inhibitor. In certain embodiments, an ITK inhibitor and a BET inhibitor are administered in combination with an additional therapeutic agent for the treatment of a hematological malignancy. In certain embodiments, a BTK inhibitor (e.g. ibrutinib) and a BET inhibitor are administered in combination with an additional therapeutic agent for the treatment of a hematological malignancy. In some embodiments, the additional therapeutic agent is an EZH2 inhibitor, or an HDAC inhibitor. In some embodiments, the additional therapeutic agent is selected from a chemotherapeutic agent, a biologic agent, radiation therapy, bone marrow transplant or surgery.

In some embodiments, the third therapeutic agent is an EZH2 inhibitor. EZH2 (histone-lysine N-methyltransferase EZH2) is a gene silencer which methylates Lys 9 (H3K9me) and Lys 27 (H3K27me2) of histone H3, leading to transcriptional repression of the affected target genes as well as to chromatin condensation. Inhibitors of EZH2 include, but are not limited to, EPZ-6438 (E-7438) and EPZ-005687 from Epizyme®, GSK126, GSK343, and GSK2816126 from GlaxoSmithKline, UNC-1999, UNC2399, UNC2400, 3-deazaneplanocin A (DZNep), Ell, MC1948 and MC1945.

In some embodiments, the third therapeutic agent is an HDAC inhibitor. In some embodiments, the HDAC inhibitors are classified into pan-HDAC inhibitors and specific HDAC inhibitors. In some embodiments, the HDAC inhibitors include, but are not limited to, hydroxamates such as TSA, SAHA, zolinza, vornostat, CBHA, LAQ-824, PDX-101, LBH-589, ITF2357, and PCI-24781; cyclic peptides such as depsipeptide (FK-228); aliphatic acids such as valproic acid, phenyl butyrate, butyrate, and AN-9; and benzamides such as MS-275 and MGCD0103.

In some embodiments, the third therapeutic agent is selected from among a chemotherapeutic agent, a biologic agent, radiation therapy, bone marrow transplant or surgery. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

### Pharmaceutical Compositions and Formulations

Disclosed herein, in certain embodiments, are pharmaceutical compositions and formulations comprising: (a) BTK inhibitor; (b) a BET inhibitor; and (c) a pharmaceutically-acceptable excipient. In some embodiments, the BTK inhibitor is ibrutinib. In some embodiments, the BET inhibitor is selected from among CPI-0610, DUAL946, GSK525762, I-BET151, JQ1, OTX015, PFI-1, RVX-208, RVX2135, TEN-010, or a combination thereof. In some embodiments, the BET inhibitor is I-BET151, JQ1, OTX015, or a combination thereof. In some embodiments, the BET inhibitor is I-BET151. In some embodiments, the BET inhibitor is JQ1. In some embodiments, the BET inhibitor is OTX015. In some embodiments, the BTK inhibitor is ibrutinib and the BET inhibitor is selected from among CPI-0610, DUAL946, GSK525762, I-BET151, JQ1, OTX015, PFI-1, RVX-208, RVX2135, TEN-010, or a combination thereof. In some embodiments, the BTK inhibitor is ibrutinib and the BET inhibitor is I-BET151, JQ1, OTX015, or a combination thereof. In some embodiments, the BTK inhibitor is ibrutinib and the BET inhibitor is I-BET151. In some embodiments, the BTK inhibitor is ibrutinib and the BET inhibitor is JQ1. In some embodiments, the BTK inhibitor is ibrutinib and the BET inhibitor is OTX015.

In some embodiments, the combination of a BTK inhibitor and a BET inhibitor exert a synergistic effect. In some embodiments, the combination of a BTK inhibitor and a BET inhibitor exert an additive effect. In some embodiments, the combination of a BTK inhibitor and a BET inhibitor exert an antagonistic effect. In some embodiments, the combination of a BTK inhibitor and a BET inhibitor sensitize cells to the BTK inhibitor. In some embodiments, the combination of a BTK inhibitor and a BET inhibitor exert no effect on the cells. In some embodiments, synergism is further subdivided into very strong synergism, strong synergism, synergism, moderate synergism, and slight synergism. In some embodiments, the combination of a BTK inhibitor and a BET inhibitor exert a very strong synergistic effect, a strong synergistic effect, a synergistic effect, a moderate synergistic effect, a slight synergistic effect, or a combination thereof. In some embodiments, the combination of a BTK inhibitor and a BET inhibitor exert a very strong synergistic effect. In some embodiments, the BTK inhibitor is ibrutinib.

In some embodiments, the combination of ibrutinib and a BET inhibitor exert a synergistic effect. In some embodiments, the combination of ibrutinib and a BET inhibitor exert an additive effect. In some embodiments, the combination of ibrutinib and a BET inhibitor exert an antagonistic effect. In some embodiments, the combination of ibrutinib and a BET inhibitor sensitize cells to ibrutinib. In some embodiments, the combination of ibrutinib and a BET inhibitor exert no effect on the cells. In some embodiments, synergism is further subdivided into very strong synergism, strong synergism, synergism, moderate synergism, and slight synergism. In some embodiments, the combination of ibrutinib and a BET inhibitor exert a very strong synergistic effect, a strong synergistic effect, a synergistic effect, a moderate synergistic effect, a slight synergistic effect, or a combination thereof. In some embodiments, the combination of ibrutinib and a BET inhibitor exert a very strong synergistic effect.

In some embodiments, a combination index (CI) value is used to indicate the behavior of the combination of a BTK inhibitor (e.g. ibrutinib) and a BET inhibitor. In some embodiments, CI<1 indicates a synergistic effect. In some embodiments, CI=1 indicates an addictive effect. In some embodiments, CI>1 indicates an antagonistic effect. In some embodiments, synergism is further subdivided into very strong synergism, strong synergism, synergism, moderate synergism, and slight synergism. In some embodiments, the CI value for a very strong synergism is at most 0.1, or less. In some embodiments, the CI value for a strong synergism is from about 0.1 to about 0.9, about 0.1 to about 0.5, or about 0.1 to about 0.3. In some embodiments, the CI value for a synergism is from about 0.1 to about 0.9, about 0.2 to about 0.8, or about 0.3 to about 0.7. In some embodiments, the CI value for a moderate synergism is from about 0.1 to about 0.9, about 0.3 to about 0.9, or about 0.7 to about 0.85. In some embodiments, the CI value for a slight synergism is from about 0.1 to about 0.9, about 0.5 to about 0.9, or about 0.85 to about 0.9.

In some embodiments, the combination of an ITK inhibitor and a BET inhibitor exert a synergistic effect, an additive effect, or an antagonistic effect. In some embodiments, the combination of an ITK inhibitor and a BET inhibitor sensitize cells to the ITK inhibitor. In some embodiments, the combination of an ITK inhibitor and a BET inhibitor exert no effect on the cells. In some embodiments, the combination of a TEC inhibitor and a BET inhibitor exert a synergistic effect, an additive effect, or an antagonistic effect. In some embodiments, the combination of a TEC inhibitor and a BET inhibitor sensitize cells to the TEC inhibitor. In some embodiments, the combination of a TEC inhibitor and a BET inhibitor exert no effect on the cells.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

A pharmaceutical composition, as used herein, refers to a mixture of a compound described herein, such as, for example, ibrutinib and A BET inhibitor, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of ibrutinib and A BET inhibitor, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per mL, dL, or L of blood serum, absorbed into the bloodstream after administration. As used herein, measurable plasma concentrations are typically measured in ng/ml or μg/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Dosage Forms

The compositions described herein can be formulated for administration to a subject via any conventional means including, but not limited to, oral, parenteral (e.g., intravenous, subcutaneous, or intramuscular), buccal, intranasal, rectal or transdermal administration routes. In some embodiments, the composition is formulated for administration in a combined dosage form. In some embodiments, the composition is formulated for administration in a separate dosage forms. As used herein, the term “subject” is used to mean an animal, preferably a mammal, including a human or non-human. The terms “individual(s)”, “subject(s)” and “patient(s)” are used interchangeably herein, and mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

Moreover, the pharmaceutical compositions described herein, which include ibrutinib and/or a BET inhibitor can be formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations described herein may be administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of ibrutinib and/or a BET inhibitor, with one or more pharmaceutical excipients to form a bulk blend composition. When referring to these bulk blend compositions as homogeneous, it is meant that the particles of ibrutinib and/or a BET inhibitor, are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of ibrutinib and/or a BET inhibitor. In another embodiment, some or all of the particles of ibrutinib and/or a BET inhibitor, are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release the compound of ibrutinib and/or a BET inhibitor, from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

In other embodiments, one or more layers of the pharmaceutical formulation are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet. In some embodiments, the film coating can provide a delayed release of ibrutinib or the second agent, from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

A capsule may be prepared, for example, by placing the bulk blend of the formulation of ibrutinib or the second agent, described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of ibrutinib and/or a BET inhibitor, and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with ibrutinib and/or a BET inhibitor, which sufficiently isolate the compound of any of ibrutinib or a BET inhibitor, from other non-compatible excipients. Materials compatible with compounds of any of ibrutinib or a BET inhibitor, are those that delay the release of the compounds of any of ibrutinib or a BET inhibitor, in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose S R, Methocel®-E, Opadry Y S, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® 512.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

Microencapsulated compounds of any of ibrutinib or a BET inhibitor, may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

In one embodiment, the particles of compounds of any of ibrutinib or a BET inhibitor, are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In other embodiments, the solid dosage formulations of the compounds of any of ibrutinib and/or a BET inhibitor, are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In other embodiments, a powder including the formulations with a compound of any of ibrutinib and/or a BET inhibitor, described herein, may be formulated to include one or more pharmaceutical excipients and flavors. Such a powder may be prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions described herein to achieve delivery to the lower gastrointestinal tract. In some embodiments the polymers described herein are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH>7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions; Poly Vinyl Acetate Phthalate (PVAP). PVAP dissolves in pH>5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

In other embodiments, the formulations described herein, which include ibrutinib and/or a BET inhibitor, are delivered using a pulsatile dosage form. A pulsatile dosage form is capable of providing one or more immediate release pulses at predetermined time points after a controlled lag time or at specific sites. Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

In some embodiments, pharmaceutical formulations are provided that include particles of ibrutinib and/or a BET inhibitor, described herein and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents suitable for the aqueous suspensions and dispersions described herein are known in the art and include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.001% to about 1.0% the volume of the aqueous dispersion. In another embodiment, the aqueous liquid dispersion can comprise a sweetening agent or flavoring agent in a concentration ranging from about 0.005% to about 0.5% the volume of the aqueous dispersion. In yet another embodiment, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical formulations described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563, each of which is specifically incorporated by reference.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein. The amounts of such additives can be readily determined by one skilled in the art, according to the particular properties desired.

### Intranasal Formulations

Intranasal formulations are known in the art and are described in, for example, U.S. Pat. Nos. 4,476,116, 5,116,817 and 6,391,452, each of which is specifically incorporated by reference. Formulations that include ibrutinib and/or A BET inhibitor, which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. The nasal dosage form should be isotonic with nasal secretions.

For administration by inhalation described herein may be in a form as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

### Buccal Formulations

Buccal formulations may be administered using a variety of formulations known in the art. For example, such formulations include, but are not limited to, U.S. Pat. Nos. 4,229,447, 4,596,795, 4,755,386, and 5,739,136, each of which is specifically incorporated by reference. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with ibrutinib and/or A BET inhibitor, and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

### Transdermal Formulations

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety.

The transdermal dosage forms described herein may incorporate certain pharmaceutically acceptable excipients which are conventional in the art. In one embodiments, the transdermal formulations described herein include at least three components: (1) a formulation of a compound of ibrutinib and A BET inhibitor; (2) a penetration enhancer; and (3) an aqueous adjuvant. In addition, transdermal formulations can include additional components such as, but not limited to, gelling agents, creams and ointment bases, and the like. In some embodiments, the transdermal formulation can further include a woven or non-woven backing material to enhance absorption and prevent the removal of the transdermal formulation from the skin. In other embodiments, the transdermal formulations described herein can maintain a saturated or supersaturated state to promote diffusion into the skin.

Formulations suitable for transdermal administration of compounds described herein may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of ibrutinib and A BET inhibitor. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

### Injectable Formulations

Formulations that include a compound of ibrutinib and/or A BET inhibitor, suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

### Other Formulations

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Dosing and Treatment Regiments

In some embodiments, the amount of ibrutinib that is administered in combination with a BET inhibitor is from 10 mg/day up to, and including, 1000 mg/day. In some embodiments, the amount of ibrutinib that is administered is from about 40 mg/day to 70 mg/day. In some embodiments, the amount of Ibrutinib that is administered per day is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the amount of brutinib that is administered is about 40 mg/day. In some embodiments, the amount of ibrutinib that is administered is about 50 mg/day. In some embodiments, the amount of ibrutinib that is administered is about 60 mg/day. In some embodiments, the amount of ibrutinib that is administered is about 70 mg/day.

In some embodiments, the amount of a BET inhibitor that is administered in combination with ibrutinib is from 0.01 μM to, and including, 100 μM. In some embodiments, the amount of a BET inhibitor is from about 0.01 μM to about 100 μM.

In some embodiments, ibrutinib is administered once per day, twice per day, or three times per day. In some embodiments, ibrutinib is administered once per day. In some embodiments, a BET inhibitor is administered once per day, twice per day, or three times per day. In some embodiments, a BET inhibitor is administered once per day. In some embodiments, Ibrutinib and a BET inhibitor are co-administered (e.g., in a single dosage form), once per day.

In some embodiments, the compositions disclosed herein are administered for prophylactic, therapeutic, or maintenance treatment. In some embodiments, the compositions disclosed herein are administered for therapeutic applications. In some embodiments, the compositions disclosed herein are administered for therapeutic applications. In some embodiments, the compositions disclosed herein are administered as a maintenance therapy, for example for a patient in remission.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, the severity of the disease, the identity (e.g., weight) of the subject or host in need of treatment, but can nevertheless be routinely determined in a manner known in the art according to the particular circumstances surrounding the case, including, e.g., the specific agent being administered, the route of administration, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. Compounds exhibiting high therapeutic indices are preferred. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include ibrutinib, optionally in a composition or in combination with a BET inhibitor as disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

These examples are provided for illustrative purposes only and not to limit the scope of the claims provided herein.

### Example 1

### Combined Drug Treatment for Cell Viability

Different DLBCL cell lines were tested in vitro to determine the synergistic and antagonistic effect of ibrutinib with BET inhibitors.

The DLBCL cell lines used during the experiments included TMD8, OCI-LY3, OCI-LY10, U-2932 and SU-DHL-2.

DLBCL cells at either 1×10⁴ cells or 2×10⁴ cells were plated onto each well of a 96-W plate (Table 1).

TABLE 1

| Cells | Medium | cells/well (200 ul) | cells/ml |
|---|---|---|---|
| TMD8 | R-10 + S | 10000 | 50000 |
| OCI-LY-3 | IM-10 | 10000 | 50000 |
| OCI-LY-10 | IM-10 | 10000 | 50000 |
| U-2932 | R-10 | 20000 | 100000 |
| SU-DHL-2 | R-10 | 20000 | 100000 |

Ibrutinib at 10000, 2000, 400, 80, 16, 3.2, 0.64, 0.128, 0.0256, and 0 nM concentrations were used during the experiments. The concentrations of the BET inhibitors, I-BET151, JQ1 and OTX015, are shown in Table 2. The stock solution for ibrutinib was prepared at 20 mM concentration. The stock solutions for the BET inhibitors, I-BET-151, JQ1 and OTX015, were each prepared at 50 mM concentration.

TABLE 2

| | TMD8 | LY-3 | LY-10 | U-2932 | SU-DHL-2 |
|---|---|---|---|---|---|
| I-BET151 | 200 nM | 5 uM | 5 uM | 200 nM | 2 uM |
| JQ1 | 20 nM | 1 uM | 1 uM | 20 nM | 100 nM |
| OTX015 | 100 nM | 1 uM | 1 uM | 50 nM | 1 uM |

To each well of a 96-W plate was added 100 μL ibrutinib (2× of target concentration; diluted using appropriate cell medium for each cell line), 50 μL BET inhibitor (4× of target concentration), and 50 μL of cells (4× target concentration). The 96-W plate was then incubated for 3 days. Cell viability was examined using a CellTiter-Glo assay.

### CellTiter-Glo Assay

A 40 μL of CellTiter-Glo reagent was added directly into each well of the 96-W plate. The plate was then shaken on a Shaker (Labsystem Wellmix) at speed 5 for 10-20 min at room temperature. Next, about 100 μL of the mixed medium was transferred to a white, non-transparent, flat bottom 96-W plate for assaying. A Flexstation 3 luminometer was used for detecting and measuring the luminescent signals. Measurements were taken at room temperature.

CellTiter-Glow reagents were thawed prior to use. Cells pre-plated onto a second 96-W plate and incubated at room temperature for 30 minutes were used for calibration purposes.

Table 3 indicates the experimental design layout on the 96-W plate.

TABLE 3

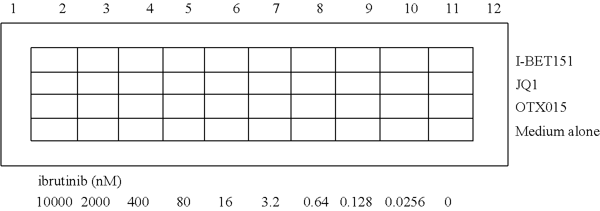

Tables 4-8 illustrate the luminescent signals for each cell line.

TABLE 4
TMD8

| 2 | 3 | 4 | 5 | 6 | 7 | 8 | 9 | 10 | 11 | |
|---|---|---|---|---|---|---|---|---|---|---|
| 6995.257 | 11183.45 | 11191.36 | 11526.1 | 11328.42 | 11655.25 | 12722.72 | 13950.98 | 14177.65 | 53526.63 | I-BET151 |
| 9818.137 | 15571.96 | 15282.03 | 15297.84 | 16312.6 | 16468.11 | 16842.39 | 18911.45 | 18249.87 | 59694.27 | JQ1 |
| 7055.879 | 10927.78 | 9301.531 | 9472.854 | 9272.538 | 9773.329 | 9847.13 | 11191.36 | 11004.22 | 47319.47 | OTX015 |
| 10819.72 | 23421.2 | 25674.76 | 28263.05 | 27190.31 | 31383.77 | 29180.29 | 32828.16 | 32496.06 | 84399.08 | Medium alone |
| ibrutinib (nM) | | | | | | | | | | |
| 10000 | 2000 | 400 | 80 | 16 | 3.2 | 0.64 | 0.128 | 0.0256 | 0 | |

TABLE 5
OCI-LY-10

| 2 | 3 | 4 | 5 | 6 | 7 | 8 | 9 | 10 | 11 | |
|---|---|---|---|---|---|---|---|---|---|---|
| 21920.51 | 22475.36 | 21781.14 | 21683.85 | 23382.58 | 31834.18 | 38594.93 | 38721.15 | 41108.85 | 43633.29 | I-BET151 |
| 25346.91 | 24579.06 | 24502.8 | 24844.65 | 26164.72 | 39783.52 | 52166.4 | 47932.71 | 45492.43 | 41458.59 | JQ1 |
| 23945.32 | 24952.47 | 25433.68 | 25880.72 | 29764.67 | 33872.14 | 40790.66 | 42647.18 | 42481.51 | 45142.68 | OTX015 |
| 24826.24 | 24452.84 | 23784.91 | 23511.43 | 27324.38 | 51190.81 | 63983.91 | 68196.56 | 61548.88 | 58403.86 | Medium alone |
| ibrutinib (nM) | | | | | | | | | | |
| 10000 | 2000 | 400 | 80 | 16 | 3.2 | 0.64 | 0.128 | 0.0256 | 0 | |

TABLE 6
OCI-LY-3

| 2 | 3 | 4 | 5 | 6 | 7 | 8 | 9 | 10 | 11 | |
|---|---|---|---|---|---|---|---|---|---|---|
| 14424.2 | 13151.56 | 15392.46 | 13899.4 | 14914.89 | 13849.54 | 15156.3 | 12962.63 | 15418.7 | 14156.55 | I-BET151 |
| 21414.57 | 24841.54 | 22621.62 | 21944.63 | 21821.3 | 22419.57 | 23078.2 | 22582.26 | 21973.49 | 20530.28 | JQ1 |
| 14418.95 | 15793.94 | 15615.51 | 14290.38 | 13776.07 | 14043.72 | 13925.64 | 12918.02 | 14387.47 | 12422.08 | OTX015 |
| 63477.51 | 55749.79 | 64472.02 | 68972.2 | 64655.7 | 66789.02 | 63322.7 | 59441.78 | 63957.7 | 52879.12 | Medium alone |
| ibrutinib (nM) | | | | | | | | | | |
| 10000 | 2000 | 400 | 80 | 16 | 3.2 | 0.64 | 0.128 | 0.0256 | 0 | |

TABLE 7
U2932

| 2 | 3 | 4 | 5 | 6 | 7 | 8 | 9 | 10 | 11 | |
|---|---|---|---|---|---|---|---|---|---|---|
| 32015.12 | 42568.25 | 44468.08 | 43659.14 | 43980.61 | 43472.05 | 42565.61 | 44167.69 | 45116.29 | 50367.82 | I-BET151 |
| 33551.32 | 44099.18 | 44892.31 | 49232.14 | 46417.97 | 47334.95 | 46610.32 | 54454.69 | 52636.54 | 55050.2 | JQ1 |
| 33140.26 | 44022.77 | 48570.75 | 44565.57 | 49095.12 | 45013.52 | 50299.31 | 51182.03 | 53427.04 | 55416.46 | OTX015 |
| 37935.94 | 46858.01 | 46009.54 | 49777.58 | 51023.93 | 53031.79 | 53722.16 | 57150.28 | 63123.8 | 65814.13 | Medium alone |
| ibrutinib (nM) | | | | | | | | | | |
| 10000 | 2000 | 400 | 80 | 16 | 3.2 | 0.64 | 0.128 | 0.0256 | 0 | |

TABLE 8
SU-DHL-2

| 2 | 3 | 4 | 5 | 6 | 7 | 8 |
|---|---|---|---|---|---|---|
| 585.813 | 717.161 | 798.597 | 922.065 | 843.256 | 809.105 | 704.027 |
| 3706.648 | 29411.5 | 23679.47 | 19823.08 | 20485.07 | 25589.27 | 19308.19 |
| 782.836 | 1266.197 | 1190.015 | 1179.507 | 1276.705 | 1166.373 | 1116.46 |
| 111485.8 | 154426.1 | 159908.6 | 164140.7 | 155353.5 | 161613.5 | 159399 |
| ibrutinib (nM) | | | | | | |
| 10000 | 2000 | 400 | 80 | 16 | 3.2 | 0.64 |

| 9 | 10 | 11 | |
|---|---|---|---|
| 835.375 | 714.534 | 793.343 | I-BET151 |
| 19174.22 | 22662.83 | 17739.9 | JQ1 |
| 1087.564 | 1016.636 | 945.707 | OTX015 |
| 146159.1 | 141202 | 143348.2 | Medium alone |
| 0.128 | 0.0256 | 0 | |

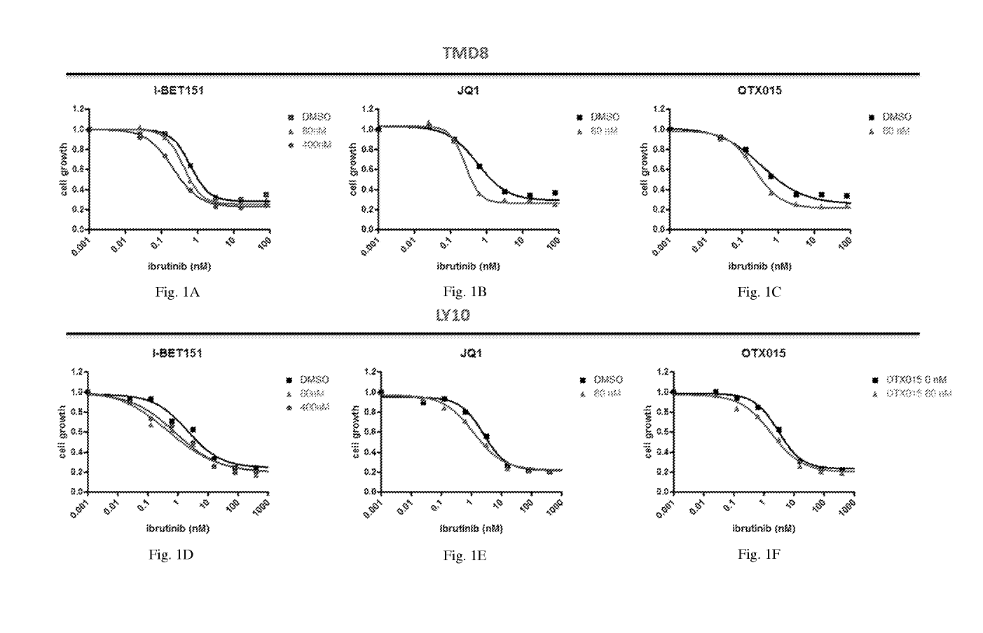

The luminescent measurements were subsequently process and analyzed to derive combination index (CI) for each combination of ibrutinib and a BET inhibitor at each cell line. CI is a quantitative description of the interaction property of the combination of two drugs. In general, the combination is described as synergistic (CI<1), additive (CI=1), or antagonistic (CI>1). Synergism is further separated into very strong synergism (<0.1), strong synergism (0.1-0.3), synergism (0.3-0.7), moderate synergism (0.7-0.85), and slight synergism (0.85-0.9). Tables 9-23 illustrate the CI values for each ibrutinib and BET inhibitor combination in each cell line. Tables 9-11 illustrate the CI values for each ibrutinib and BET inhibitor combination in TMD8 cell line. Tables 12-14 illustrate the CI values for each ibrutinib and BET inhibitor combination in OCI-LY10 cell line. Tables 15-17 illustrate the CI values for each ibrutinib and BET inhibitor combination in OCI-LY3 cell line. Tables 18-20 illustrate the CI values for each ibrutinib and BET inhibitor combination in U2932 cell line. Tables 21-23 illustrate the CI values for each ibrutinib and BET inhibitor combination in SU-DHL-2 cell line. CI values were calculated for all cell lines, however before CI values were evaluated, cell growth curves were analyzed to see if the second compound enhanced ibrutinib sensitivity in each cell line. For those cell lines in which ibrutinib sensitivity was not enhanced by the second compound, CI values were not considered. Growth curves indicated that BET inhibitors sensitized the TMD8 cells and LY10 cells to ibrutinib (FIGS. 1A-1F). The gray regions in Tables 9-14 indicate synergism for the respective ibrutinib and BET inhibitor combinations.

### Tables 9-11: TMD8 Cell Line

TABLE 9
ibrutinib + I-BET151 Combination

| Ibrutinib | I-BET151 | CI |
|---|---|---|
| 0.0256 | 200 | 0.104 |
| 0.128 | 200 | 0.102 |
| 0.64 | 200 | 0.091 |
| 3.2 | 200 | 0.081 |
| 16 | 200 | 0.079 |
| 80 | 200 | 0.08 |
| 400 | 200 | 0.078 |
| 2000 | 200 | 0.078 |
| 10000 | 200 | 0.045 |

TABLE 10
ibrutinib + JQ1 Combination

| Ibrutinib | JQ1 | CI |
|---|---|---|
| 0.0256 | 20 | 0.072 |
| 0.128 | 20 | 0.077 |
| 0.64 | 20 | 0.062 |
| 3.2 | 20 | 0.06 |
| 16 | 20 | 0.059 |
| 80 | 20 | 0.053 |
| 400 | 20 | 0.056 |
| 2000 | 20 | 0.082 |
| 10000 | 20 | 0.025 |

TABLE 11
ibrutinib + OTX015 Combination

| Ibrutinib | OTX015 | CI |
|---|---|---|
| 0.0256 | 100 | 0.085 |
| 0.128 | 100 | 0.088 |
| 0.64 | 100 | 0.071 |
| 3.2 | 100 | 0.07 |
| 16 | 100 | 0.064 |
| 80 | 100 | 0.067 |
| 400 | 100 | 0.065 |
| 2000 | 100 | 0.084 |
| 10000 | 100 | 0.042 |

### Tables 12-14: OCI-LY-10 Cell Line

TABLE 12
ibrutinib + I-BET151 Combination

| Ibrutinib | I-BET151 | CI |
|---|---|---|
| 0.0256 | 5000 | 0.896 |
| 0.128 | 5000 | 0.807 |
| 0.64 | 5000 | 0.812 |
| 3.2 | 5000 | 0.61 |
| 16 | 5000 | 0.418 |
| 80 | 5000 | 0.419 |
| 400 | 5000 | 0.616 |
| 2000 | 5000 | 1.804 |
| 10000 | 5000 | 6.633 |

TABLE 13
ibrutinib + JQ1 Combination

| Ibrutinib | JQ1 | CI |
|---|---|---|
| 0.0256 | 1000 | 0.786 |
| 0.128 | 1000 | 0.973 |
| 0.64 | 1000 | 1.653 |
| 3.2 | 1000 | 0.588 |
| 16 | 1000 | 0.221 |
| 80 | 1000 | 0.272 |
| 400 | 1000 | 0.614 |
| 2000 | 1000 | 2.416 |
| 10000 | 1000 | 13.376 |

TABLE 14
ibrutinib + OTX015 Combination

| Ibrutinib | OTX015 | CI |
|---|---|---|
| 0.0256 | 1000 | 1.327 |
| 0.128 | 1000 | 1.364 |
| 0.64 | 1000 | 1.059 |
| 3.2 | 1000 | 0.423 |
| 16 | 1000 | 0.279 |
| 80 | 1000 | 0.249 |
| 400 | 1000 | 0.66 |
| 2000 | 1000 | 2.542 |
| 10000 | 1000 | 9.881 |

### Tables 15-17: OCI-LY-3 Cell Line

TABLE 15
ibrutinib + I-BET151 Combination

| Ibrutinib | I-BET151 | CI |
|---|---|---|
| 0.0256 | 5000 | 0.26 |
| 0.128 | 5000 | 0.192 |
| 0.64 | 5000 | 0.253 |
| 3.2 | 5000 | 0.216 |
| 16 | 5000 | 0.246 |
| 80 | 5000 | 0.217 |
| 400 | 5000 | 0.26 |
| 2000 | 5000 | 0.197 |
| 10000 | 5000 | 0.231 |

TABLE 16
ibrutinib + JQ1 Combination

| Ibrutinib | JQ1 | CI |
|---|---|---|
| 0.0256 | 1000 | 0.721 |
| 0.128 | 1000 | 0.747 |
| 0.64 | 1000 | 0.768 |
| 3.2 | 1000 | 0.74 |
| 16 | 1000 | 0.714 |
| 80 | 1000 | 0.719 |
| 400 | 1000 | 0.748 |
| 2000 | 1000 | 0.848 |
| 10000 | 1000 | 0.697 |

TABLE 17
ibrutinib + OTX015 Combination

| Ibrutinib | OTX015 | CI |
|---|---|---|
| 0.0256 | 1000 | 0.516 |
| 0.128 | 1000 | 0.475 |
| 0.64 | 1000 | 0.503 |
| 3.2 | 1000 | 0.506 |
| 16 | 1000 | 0.499 |
| 80 | 1000 | 0.513 |
| 400 | 1000 | 0.549 |
| 2000 | 1000 | 0.554 |
| 10000 | 1000 | 0.516 |

### Tables 18-20: U2932 Cell Line

TABLE 18
ibrutinib + I-BET151 Combination

| Ibrutinib | I-BET151 | CI |
|---|---|---|
| 0.0256 | 2000 | 4.905 |
| 0.128 | 2000 | 4.484 |
| 0.64 | 2000 | 3.871 |
| 3.2 | 2000 | 4.207 |
| 16 | 2000 | 4.423 |
| 80 | 2000 | 4.35 |
| 400 | 2000 | 5.122 |
| 2000 | 2000 | 5.036 |
| 10000 | 2000 | 1.688 |

TABLE 19
ibrutinib + JQ1 Combination

| Ibrutinib | JQ1 | CI |
|---|---|---|
| 0.0256 | 100 | 3.644 |
| 0.128 | 100 | 5.245 |
| 0.64 | 100 | 1.366 |
| 3.2 | 100 | 1.531 |
| 16 | 100 | 1.373 |
| 80 | 100 | 2.902 |
| 400 | 100 | 1.678 |
| 2000 | 100 | 3.141 |
| 10000 | 100 | 0.444 |

TABLE 20
ibrutinib + OTX015 Combination

| Ibrutinib | OTX015 | CI |
|---|---|---|
| 0.0256 | 1000 | 30.578 |
| 0.128 | 1000 | 19.651 |
| 0.64 | 1000 | 16.748 |
| 3.2 | 1000 | 7.134 |
| 16 | 1000 | 13.743 |
| 80 | 1000 | 6.779 |
| 400 | 1000 | 15.611 |
| 2000 | 1000 | 8.278 |
| 10000 | 1000 | 1.601 |

### Tables 21-23: SU-DHL-2 Cell Line

TABLE 21
ibrutinib + I-BET151 Combination

| Ibrutinib | I-BET151 | CI |
|---|---|---|
| 0.0256 | 200 | 0.044 |
| 0.128 | 200 | 0.048 |
| 0.64 | 200 | 0.044 |
| 3.2 | 200 | 0.048 |
| 16 | 200 | 0.049 |
| 80 | 200 | 0.051 |
| 400 | 200 | 0.047 |
| 2000 | 200 | 0.044 |
| 10000 | 200 | 0.04 |

TABLE 22
ibrutinib + JQ1 Combination

| Ibrutinib | JQ1 | CI |
|---|---|---|
| 0.0256 | 20 | 0.175 |

TABLE 23
ibrutinib + OTX015 Combination

| Ibrutinib | OTX015 | CI |
|---|---|---|
| 0.0256 | 50 | 0.016 |
| 0.128 | 50 | 0.017 |
| 0.64 | 50 | 0.017 |
| 3.2 | 50 | 0.017 |
| 16 | 50 | 0.019 |
| 80 | 50 | 0.018 |
| 400 | 50 | 0.018 |
| 2000 | 50 | 0.019 |
| 10000 | 50 | 0.014 |

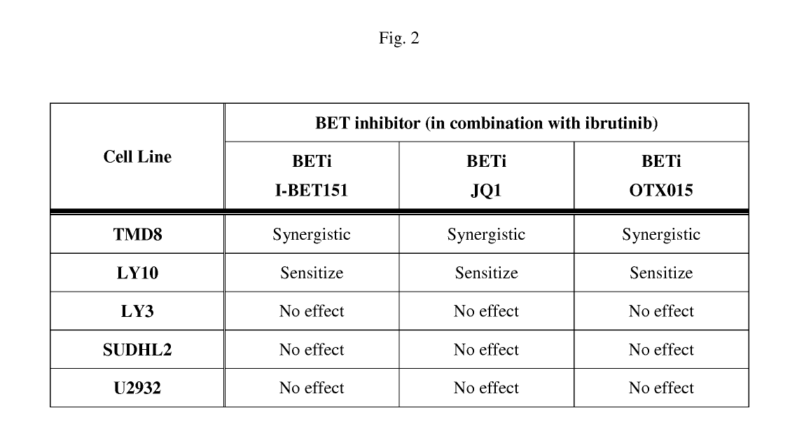

FIG. 2 illustrates the interaction property of ibrutinib in combination with the three BET inhibitors in the five cell lines. The combinations of ibrutinib with all three BET inhibitors, I-BET151, JQ1, and OTX015, were shown to exhibit synergistic effect in TMD8 cells (first row). As shown herein, the synergistic effect indicated very strong synergism (CR0.1). In OCI-LY10 cells, the combinations of ibrutinib with the three BET inhibitors were shown to sensitize OCI-LY10 cells to ibrutinib (second row). As shown herein, the sensitize effect indicated that the ibrutinib BET inhibitor combination were ranged from strong synergism to slight synergism (0.1-0.9). No effects were observed in the remaining three cell lines for all combinations of ibrutinib with the BET inhibitors (third through fifth rows). As shown herein, the no effect indicated that the ibrutinib-BET inhibitor combinations did not change the sensitivity of the cells to ibrutinib. In some embodiments, the no effect indicated that an antagonism was not observed.

### Example 2

### Combined Drug Treatment for Cell Viability

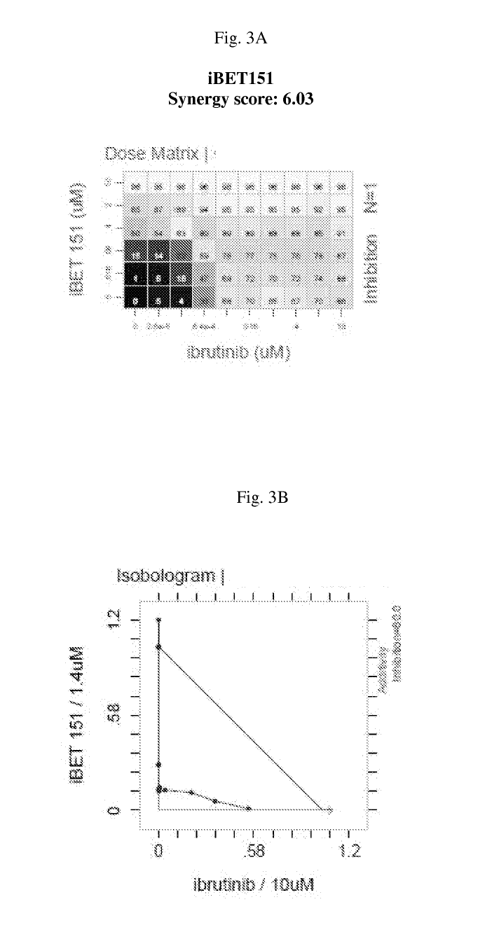

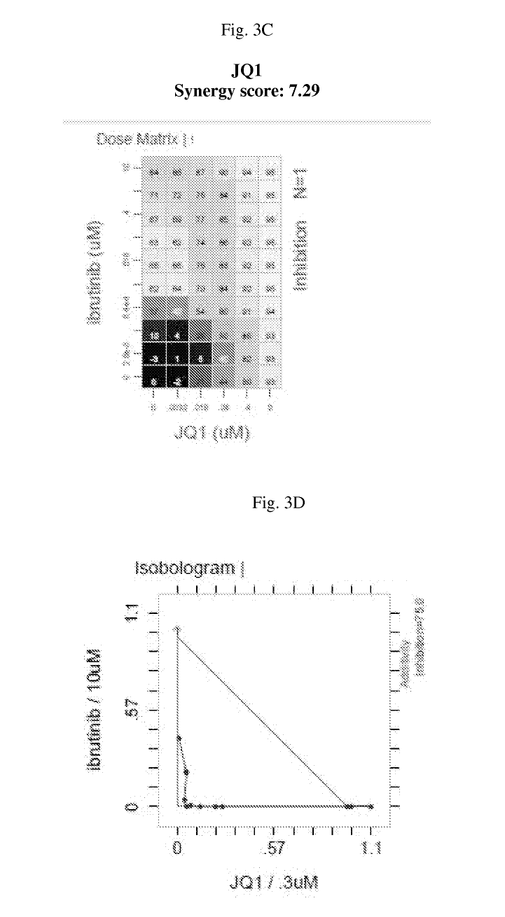

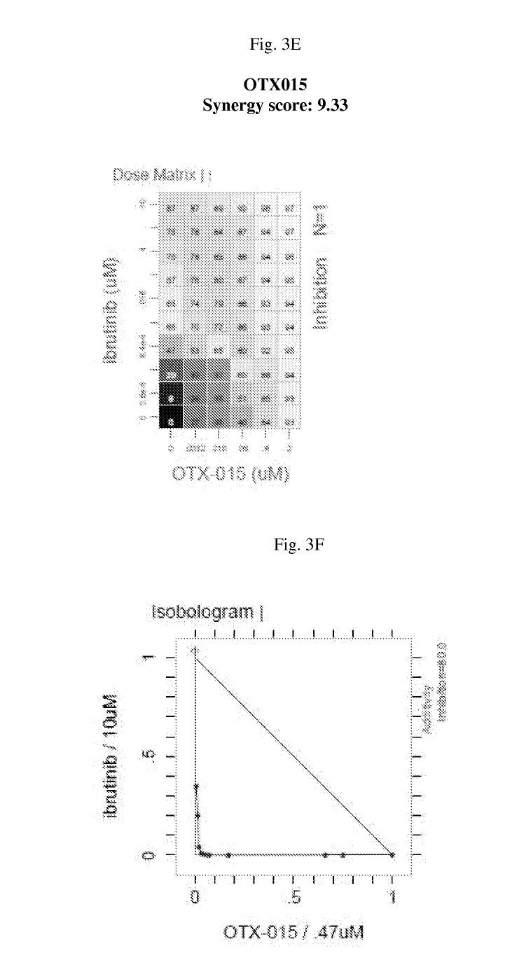

The CellTiter-Glo® luminescent cell viability assay was performed according to manufacturer's instructions. Briefly, TMD8 cells were seeded at 8,000-10,000 cells/well in a 96-well plate in the presence of BET inhibitor or ibrutinib, either individually or in combination, for 3 days. Ibrutinib concentrations used were from 10 uM in 5-fold dilutions; iBET151 concentrations used were from 10 uM in 5-fold dilutions; JQ1 concentrations used were from 2 uM in 5 fold dilutions; OTX-015 concentrations used were from 2 uM in 5-fold dilutions. The number of viable cells in culture was determined by quantification of ATP present, which was proportional to luminescent signal detected. Synergy scores and isobolograms were calculated by the Chalice Analyzer (Horizon CombinatoRx) (FIGS. 3A-3F). Based on the isobologram (FIGS. 3B, 3D, and 3F), data points and the line falling on the left side of the diagonal line, represented two compound combinations that had synergy. A higher synergy score indicated better synergy.

### Example 3

### Combined Drug Treatment for Cell Viability

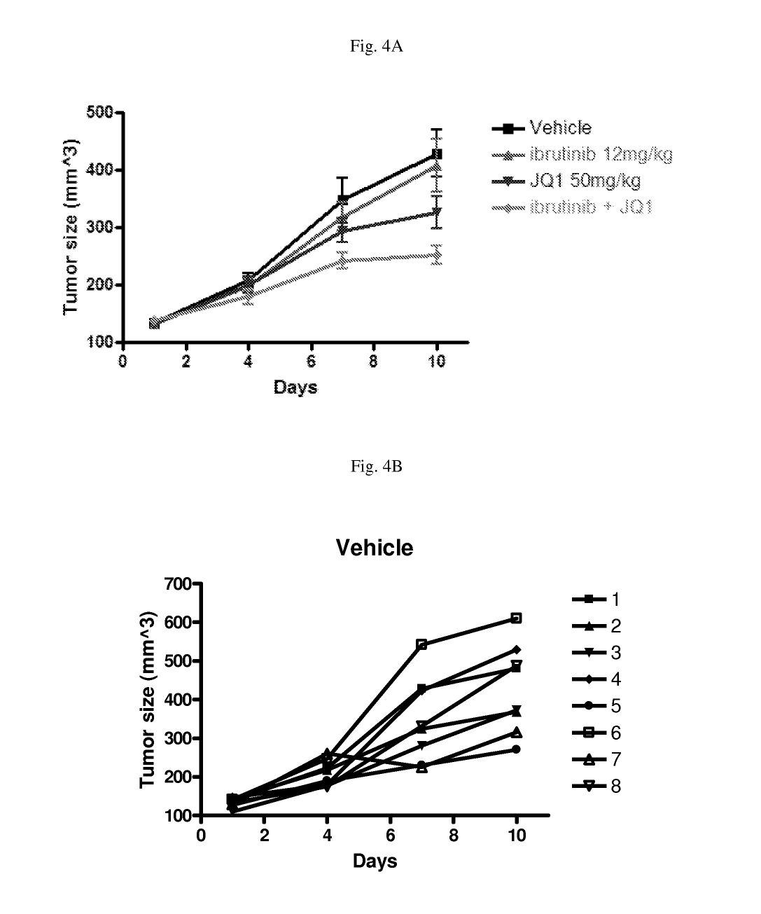

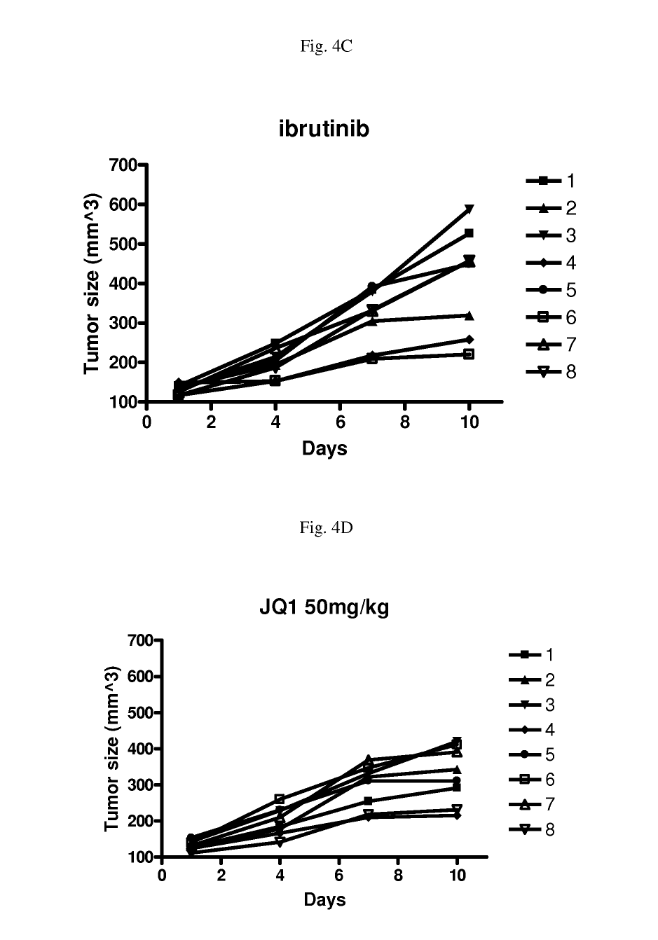

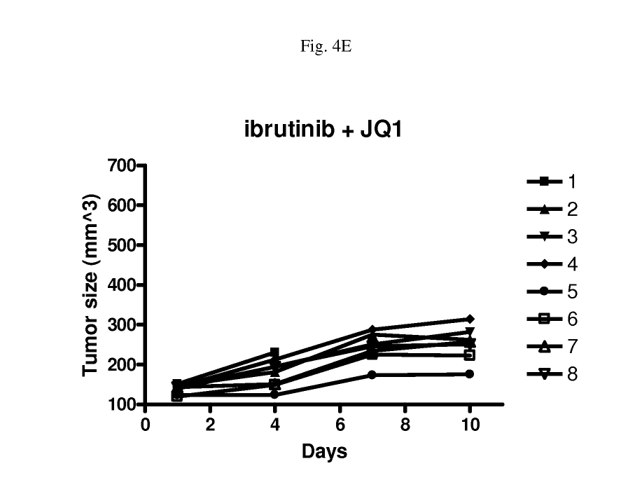

This animal study was completed under the Institutional Animal Care and Use Committee (IACUC)-approved protocols for animal welfare. BALB/c mice were subcutaneously inoculated with 5×10⁶ A20 cells. When tumors reached approximately 100 mm³ in size, mice were randomly assigned to one of the following four treatment groups: (1) vehicle, (2) ibrutinib (12 mg/kg), (3) JQ1 (50 mg/kg), or (4) the combination of ibrutinib and JQ1 (12 mg/kg ibrutinib and 50 mg/kg JQ1). Animals were treated once daily by oral gavage. Tumor volume was measured twice a week and calculated as tumor volume=(length x width²)×0.4. Tumor size over 10 days is shown for each treatment group in FIGS. 4B-4E, with average values shown in FIG. 4A. The results showed that the combination of JQ1 enhanced the growth suppression effect of ibrutinib on A20 tumors.

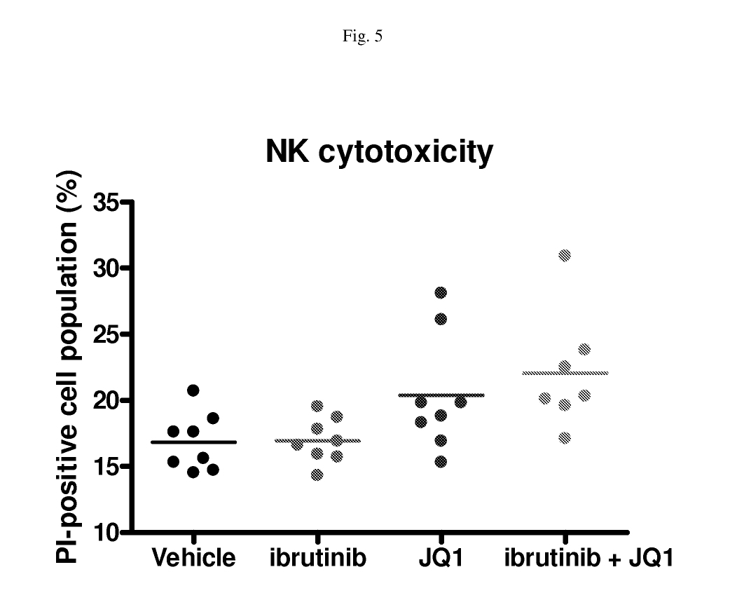

To determine the drug effect on NK cell cytotoxicity, splenocytes (effector cells) were harvested from mice after 14 days of treatment. The splenocytes were co-cultured with YAC-1 (target cells) for 4 hours. The percentage of target cell death (PI-positive cells) was analyzed by flow cytometry. The results are shown in FIG. 5, which indicate that the combination of JQ1 with ibrutinib enhanced NK cytotoxicity in the A20 model.

The examples and embodiments described herein are for illustrative purposes only and various modifications or changes suggested to persons skilled in the art are to be included within the spirit and purview of this application and scope of the appended claims.

What is claimed is:

1. A method of treating a B-cell malignancy in a subject in need thereof, comprising administering to the subject a therapeutically effective amount of a combination comprising a BTK inhibitor and a BET inhibitor.

2. The method of claim 1, wherein the combination provides a synergistic therapeutic effect compared to administration of the BTK inhibitor or the BET inhibitor alone.

3. The method of claim 1, wherein the combination sensitizes a B-cell malignancy to the BTK inhibitor.

4. The method of claim 1, wherein the BET inhibitor comprises CPI-0610, DUAL946, GSK525762, I-BET151, JQ1, OTX015, PFI-1, RVX-208, RVX2135, TEN-010, or a combination thereof.

5. The method of claim 1, wherein the BTK inhibitor is ibrutinib.

6. The method of claim 1, wherein the B-cell malignancy is acute lymphoblastic leukemia (ALL), acute myelogenous leukemia (AML), chronic myelogenous leukemia (CML), acute monocytic leukemia (AMoL), chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

7. The method of claim 6, wherein the B-cell malignancy is diffuse large B-cell lymphoma (DLBCL).

8. The method of claim 7, wherein the DLBCL is activated B-cell diffuse large B-cell lymphoma (ABC-DLBCL).

9. The method of claim 1, wherein the B-cell malignancy is a relapsed or refractory B-cell malignancy.

10. The method of claim 5, wherein ibrutinib is administered once a day, two times per day, three times per day, four times per day, or five times per day.

11. The method of claim 5, wherein ibrutinib is administered at a dosage of about 40 mg/day to about 1000 mg/day.

12. The method of claim 5, wherein ibrutinib is administered orally.

13. The method of claim 1, wherein the method further comprises administering a third therapeutic agent.

14. The method of claim 13, wherein the third therapeutic agent is a chemotherapeutic agent selected from the group consisting of chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, and a combination thereof.

15. The method of claim 1, wherein the B cell malignancy is a BTK inhibitor-resistant B cell malignancy.

16. A method of treating a B-cell malignancy associated with an elevated expression of c-MYC, comprising:
a) determining the expression level of c-MYC in a sample from an individual; and
b) administering to the individual a therapeutically effective amount of a combination comprising a BTK inhibitor and a BET inhibitor if the individual has an elevated expression level of c-MYC.

17. The method of claim 16, wherein the elevated level of c-MYC is 1-fold, 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher compared to the expression level of the reference.

18. The method of claim 16, wherein the reference level is the expression level of c-MYC in an individual who does not have a B-cell malignancy.

19. The method of claim 16, wherein the sample is a blood sample or a serum sample.

20. A pharmaceutical combination comprising:
a) a BTK inhibitor;
b) a BET inhibitor; and
c) a pharmaceutically-acceptable excipient.

21. The pharmaceutical combination of claim 20, wherein the BET inhibitor comprises CPI-0610, DUAL946, GSK525762, I-BET151, JQ1, OTX015, PFI-1, RVX-208, RVX2135, and TEN-010.

22. The pharmaceutical combination of claim 20, wherein the BTK inhibitor is ibrutinib.
